(19) United States
(12) Patent Application Publication
(10) Pub. No.: US 2017/0326072 A1
(43) Pub. Date: Nov. 16, 2017

(54) Sustained Release Formulation and Use Thereof

(71) Applicant: i-novion, Inc., Randolph, NJ (US)

(72) Inventors: Poonam R. Velagaleti, Randolph, NJ (US); Brian C. Gilger, Raleigh, NC (US); Ulrich Grau, Ueberlingen (DE); Rasidul Amin, Cary, NC (US); Santhi Abbaraju, Cary, NC (US)

(21) Appl. No.: 15/582,687

(22) Filed: Apr. 29, 2017

Related U.S. Application Data
(60) Provisional application No. 62/330,020, filed on Apr. 29, 2016.

(51) Int. Cl. A61K 9/50; A61K 47/10; A61K 47/59; A61K 9/16; C08G 65/34; C08G 63/08

(52) U.S. Cl. CPC A61K 9/5031; C08G 65/34; C08G 63/08; A61K 47/593; A61K 9/1641; A61K 47/10

(57) ABSTRACT

Provided herein are extended release polymers. In one aspect, a composition for sustained release of active ingredients comprises a block polymer having formula: PEG-PCL-PLA-PCL-PEG or PGA-PCL-PEG-PCL-PGA. The extended release block polymers modulate drug release rate based on the hydrophobicity of the PTSgel polymer irrespective of the nature of drug. PTSgel polymers are biodegradable, thermosensitive, and compatible with hydrophilic, hydrophobic, and combinations thereof, biologic or chemical active agents.

### CROSS-REFERENCE TO RELATED APPLICATIONS

This application claims priority to and the benefit of U.S. Provisional Application No. 62/330,020 filed Apr. 29, 2016, the entire disclosure of which is incorporated herein by reference.

### FIELD

The compositions and methods disclosed herein relate to thermosensitive pentablock co-polymers, biocompatible, biodegradable, and amphiphilic in nature, that disperse in aqueous medium and are specifically suitable for tunable sustained release of hydrophobic and/or hydrophilic, small molecules or biologics that are useful as therapeutics.

### BACKGROUND

Various block polymer compositions are known in the art. For example, triblock polymers such as the PCL-PEG-PCL and PLA-PEG-PLA triblock polymers comprised of polyethylene glycol (PEG) and poly(ε-caprolactone) (PCL), and polylactide (PLA) are disclosed by Cha et al., U.S. Pat. No. 5,702,717 and Lui et al. (Thermoreversible gel-sol behavior of biodegradable PCL-PEG-PCL triblock copolymer in aqueous solutions, J. Biomed. Mater. Res. B. Appl. Biomater. January, 2008, 84 (1) 165-75). The individual polymers forming the block polymer are all well-known, FDA-approved, biodegradable, and biocompatible materials.

In addition, the pentablock polymer PLA-PCL-PEG-PCL-PLA has been studied by Deng et al. (Synthesis and Characterization of Block Polymers of ε-Caprolactone and DL-Lactide Initiated by Ethylene Glycol or Poly(ethylene glycol), J. Polymer Sci., 1997, Vol 35 No. 4 703-708); Kim et al. (The Synthesis and Biodegradable behavior of PLA-PCL-PEG-PCL-PLA Multi Block Copolymer, Polymer Preprints, 2000, Vol. 49 No. 7 1557-1558). These insoluble polymers were proposed as tissue scaffolds by Huang (Polymeres Bioresorbables Dérivés de Poly(ε-caprolactone) en Ingénierie Tissulaire, Centre de Recherche surles Biopolyméres Artificiels, UMR CNRS 5473 Faculté de Pharmacie, Université Montpellier I en collaboration avec Division de Bioingénierie, Université Nationale de Singapour).

Pentablock polymer compositions described to form nanoparticles with a bioactive agent are disclosed by U.S. Pat. No. 8,551,531, PCT Publication No. WO2014/186669, and Patel et al. (Novel Thermosensitive Pentablock Copolymers for Sustained Delivery of Proteins in the Treatment of Posterior Segments Diseases, (2014) pp 1185-1200), all of which are incorporated herein by reference in their entirety. These polymers function by reducing the hydrophobicity of the compositions, thus increasing the affinity with the hydrophilic proteins and peptide active agents, to prolong the release of active agents up to 20 days. Extended release compositions need to be compatible with a variety of active agents, or combinations or active agents, for extended periods of time. Therefore, the need exists for polymer compositions that are compatible with hydrophobic, hydrophilic, or combinations of hydrophobic and hydrophilic active agents, that can deliver active agents to patients in need for extended periods of time.

### SUMMARY

The present disclosure, in one aspect, is directed to compositions of pentablock polymers useful for the sustained release of hydrophilic and/or hydrophobic active ingredients, such as biologics and small molecule drugs useful in the treatment or diagnosis of a variety of disorders or diseases.

In one aspect, a gel formulation (e.g., temperature sensitive) comprising one or more pentablock polymers is provided. For example, a pentablock polymer for preparing tunable sustained released compositions of hydrophilic and/or hydrophobic active ingredients, including biologics and small molecule drugs, can have a block polymer formula: PEG-PCL-PLA-PCL-PEG. PEG is polyethylene glycol, with an average molecular weight of about 100 to about 1000 Da, preferably about 350 to about 750 Da, more preferably about 400 to about 550 Da; PCL is poly(ε-caprolactone) with an average molecular weight of about 100 to about 3000 Da, preferably about 200 to about 2000 Da, more preferably about 400 to about 1500 Da; and PLA is polylactic acid with an average molecular weight of about 100 to about 5,000 Da, preferably about 150 to about 1,500 Da, more preferably about 250 to about 1,100 Da.

In another aspect, a pentablock polymer for preparing sustained released compositions of hydrophilic and/or hydrophobic active ingredients, including biologics and small molecule drugs, can have a block polymer formula: PGA-PCL-PEG-PCL-PGA. PEG is polyethylene glycol, with an average molecular weight of about 100 to about 1000 Da, preferably about 350 to about 750 Da, more preferably about 400 to about 550 Da. PCL is poly(ε-caprolactone) with an average molecular weight of about 100 to about 3000 Da, preferably about 200 to about 2000 Da, more preferably about 400 to about 1500 Da. PGA is polyglycolic acid with an average molecular weight of about 100 to about 5,000 Da, preferably about 150 to about 1,500 Da, more preferably about 250 to about 1,100 Da.

In certain embodiments, the PEG, PCL, PLA and/or PGA are present in an amount to increase hydrophobicity of the block polymer, thereby achieving sustained release of the active ingredient, irrespective of the hydrophilicity or hydrophobicity of the active ingredient.

In some embodiments, desired hydrophobicity can also be achieved by admixing two or more pentablock co-polymers in various proportions.

In various embodiments, the compositions of the present disclosure, comprising the block polymers disclosed herein, can bio-degrade in vivo in substantially similar time required for the release of the active ingredient, allowing for repeat injections. The polymers are dispersed in an aqueous medium and an active ingredient is being admixed, before or after adding the aqueous buffer, wherein the final concentration of the polymer is between 1% and 50%, said composition showing sustained release of the active ingredient in vitro and in vivo. The polymer concentrations can be varied between 1% and 50% to achieve different release rates. The active ingredient concentration between about 0.01% and about 50% for different release rates to be achieved. The compositions are compatible with sensitive active ingredients, such as biologics, and do not lead to significant changes in their chemical or 3-dimensional structure and, therefore, maintain full biologic activity. The compositions contain suitable therapeutic concentrations and are administered to mammals by a parenteral route or by topical application, thereby achieving biologically active levels of the active ingredient longer (e.g., 2-500 times longer) than in a standard vehicle. Further, the active ingredient retains biologic activity over the entire course of release of, e.g., up to 6 months.

In various embodiments, the pentablock polymers disclosed herein provide the surprising characteristic of tunable sustained release of various drugs, irrespective of the drug's hydrophobic or hydrophilic nature or molecular weight. Indeed, it has been surprisingly discovered that by increasing hydrophobicity of the pentablock polymer, by increasing PCL, PLA and/or PGA concentrations, and/or by decreasing PEG concentration, more prolonged, sustained release of drugs can be achieved.

### BRIEF DESCRIPTION OF THE DRAWINGS

The present technology will be further explained with reference to the attached drawings. The drawings shown are not necessarily to scale, with emphasis instead generally being placed upon illustrating the principles of the present disclosure.

FIG. 1A illustrates an FTIR spectrum of 10GH PTSgel polymer.

FIG. 1B illustrates a ¹H NMR spectrum of 10GH PTSgel polymer.

FIG. 1C illustrates a GPC chromatograph of 10GH PTSgel polymer.

FIG. 1D illustrates GPC chromatogram for reference standards (m-PEG, PCL, PLA).

FIG. 1E illustrates exemplary GPC chromatographs for various pentablock components.

FIG. 1F illustrates particle size measurement by Dynamic light spectrum (DLS) of aqueous dispersion of PTS 203GH polymer (1 mg/mL to 0.001 mg/mL in water).

FIG. 1G illustrates particle size measurement by DLS on aqueous dispersion of two polymers (PTS 210GH and PTS 1-04GH, mixed in 1:1 ratio at final concentration of 0.1 mg/mL).

FIG. 1H illustrates particle size measurement by DLS on aqueous dispersion of PTS 303 GH (composition similar to the one described in FIG. 1G but the mixture was generated by initiating synthesis with m-PEG of two different sizes).

FIG. 2 illustrates a phase diagram showing the sol-gel transition analysis of 10GH, 103GH, 113GH, and 122GH PTSgel polymers.

FIG. 3A refers to gravimetric measure of residual gel polymer following in vitro dissolution and disintegration of 10GH PTSgel.

FIG. 3B refers to GPC chromatogram of residual gel polymer and supernatant following in vitro dissolution and disintegration of 10GH PTSgel.

FIGS. 4A-4B illustrate in vitro 10 mg/mL IgG (large hydrophilic molecule) release profiles from: 101GH, 10GH, 103GH, 113GH and 122GH PTSgels, each polymer at 22.5% concentration (release modulation by change in hydrophobicity).

FIG. 5A illustrates in vitro 1 mg/mL IgG in 9.6 to 24% 10GH PTSgel release profile (release modulation by change in polymer concentration).

FIGS. 5B-5C illustrate in vitro release of Brinzolamide 2% & 4% (small hydrophobic molecule) release profiles (modulation by change in drug concentration).

FIGS. 6A-6C illustrate reduced and non-reduced SDS-PAGE size-based separations of IgG for determining IgG integrity of in vitro samples released from PTSgels.

FIG. 6D illustrates SE-HPLC analysis of IgG reference standard (left) and released sample after incubation with PTS 113GH for 28 days.

FIGS. 7A-7B illustrate in vivo IVIS imaging and quantitative profiles in mice after subcutaneous injection using NIR-IgG in 10% and 20% PTS 10GH or PTS 113GH.

FIG. 7C illustrates in vivo IVIS imaging and quantitative profiles in mice after subcutaneous injection using NIR-IgG in a mixture of two pentablock co-polymers (PTS 10GH+PTS 17GH mixed in 1:1 ratio) at 20% final polymer concentration.

FIG. 7D illustrate in vivo degradation in mice after subcutaneous injection using NIR-IgG in 10% and 20% PTS 10GH or PTS 113GH.

FIG. 7E illustrates in vivo IVIS imaging and quantitative profiles in rabbits after intracameral administration of NIR-IgG in 20% 10GH.

FIG. 8A illustrates a histological tissue analysis after treatment with a 10% and 20% 10GH and 113GH−PTSgel subcutaneous depot in mice.

FIG. 8B illustrates in vivo PTSgel safety profile following intravitreal injection in NZW Rabbits.

FIG. 8C illustrates in vivo PTSgel intracameral degradation profile following injection.

FIGS. 8D-8E illustrate in vivo PTSgel safety profile following topical eye administration.

FIG. 9 illustrates a sol-gel transition at 37° C. of 25% 102GH PTSgel solution in PBS containing 1 mg/mL pazopanib (small hydrophobic molecule).

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The present disclosure is directed to novel pentablock polymers useful for biodegradable and biocompatible sustained release drug delivery systems. The pentablock polymers described herein may be used for the substance release of biologics, or small molecules, hydrophilic and hydrophobic molecules, contained therein. Many of the pentablock polymers can exhibit reverse thermal gelation behavior, and possess good drug release characteristics. Surprisingly, by increasing hydrophobicity of the polymer composition it has been discovered that the duration of drug release can be increase, irrespective of the nature (hydrophobic/hydrophilic) and size (molecular weight) of the drug molecule. This is unexpected advantage is helpful in tuning sustained drug release.

The present disclosure is also directed to methods for fabricating the amphiphilic pentablock polymers of the present disclosure, as well as compositions comprising the biodegradable and biocompatible pentablock polymers with a hydrophilic or hydrophobic drugs, such as biologics or small molecule drugs. The present disclosure is well adapted for the administration of the hydrophilic drugs and particularly highly water-soluble biologics and small molecule hydrophilic/hydrophobic drugs. The active agents are released at a controlled rate with the corresponding biodegradation of the synthetic polymeric matrix.

The desired hydrophobicity for tunable drug release can also be achieved by admixing two or more pentablock co-polymers in various ratios.

In some embodiments, the polymers may disperse as small size particles (<1 μm in diameter, likely micellar in nature) in an aqueous medium. The particle size (in diameter) of the polymer of the present disclosure in aqueous medium as determined by DLS (Dynamic light scattering) can range from about 5 nm to about 1 μm, preferably about 7-200 nm, more preferably about 10-100 nm, and most preferably less than about 30 nm. This is a particle size that normally escapes the typical response of the body's immune system by being able to avoid phagocytosis and has enhanced permeability through biological membranes. Thus, PTSgel polymers of the present disclosure when dispersed in aqueous medium (prior to gelling), comprised of small size particles of amphiphilic in nature with high drug loading capacity of both hydrophobic and hydrophylic drugs are suitable for tunable sustained drug release of small drug molecules and biologicals through various routes of administration. In addition, the polymers described herein are biocompatible and biodegradable.

### Definitions

For convenience, certain terms employed in the specification, examples, and appended claims are collected here. Unless defined otherwise, all technical and scientific terms used herein have the same meaning as commonly understood by one of ordinary skill in the art to which this disclosure belongs.

The articles “a” and “an” are used herein to refer to one or to more than one (i.e., at least one) of the grammatical object of the article. By way of example, “an element” means one element or more than one element.

As used herein, the term “about” means within 20%, more preferably within 10% and most preferably within 5%. The term “substantially” means more than 50%, preferably more than 80%, and most preferably more than 90% or 95%.

As used herein, “a plurality of” means more than 1, e.g., 2, 3, 4, 5, 6, 7, 8, 9, 10, 11, 12, 13, 14, 15, 16, 17, 18, 19, 20, or more, e.g., 25, 30, 40, 50, 60, 70, 80, 90, 100, 200, 300, 400, 500, or more, or any integer there between.

As used herein, “administering” and similar terms mean delivering the composition to an individual being treated. Preferably, the compositions comprising the pentablock polymers of the present disclosure are administered by, e.g., parenteral, subcutaneous, intramuscular, transdermal, transmucosal, intra-articular, intrathecal, intraocular, intraperitoneal or topical routes.

As used herein, “biocompatible” refers to materials or the intermediates or end products of materials formed by solubilization hydrolysis, or by the action of biologically formed entities which can be enzymes or other products of the organism and which cause no adverse effect on the body.

As used herein, “biodegradable” means that the pentablock polymer can break down or degrade within the body to non-toxic components after all bioactive agent or diagnostic agent has been released.

As used herein, “depot” means a drug delivery liquid following injection into a warm-blooded animal which has formed a gel upon the temperature being raised to or above the LCST (lower critical solution temperature).

As used herein, “drug” or “active ingredient” or “active agent” shall refer to any biologic and/or chemical compound or substance adapted or used for a therapeutic purpose.

As used herein, “drug delivery liquid” or “drug delivery liquid having reverse thermal gelation properties” shall mean a “solution” suitable for injection into a warm-blooded animal which forms a depot upon having the temperature raised above the LCST of the polymer.

As used herein, an “effective amount” means the amount of bioactive agent or diagnostic agent that is sufficient to provide the desired local or systemic effect at a reasonable risk/benefit ratio as would attend any medical treatment or diagnostic test. This will vary depending on the patient, the disease, the treatment being effected, and the nature of the agent.

As used herein, “gel” or “PTSgel” when used in reference to the pentablock polymers and/or drug combination at a temperature at or above the LCST (see below), shall be inclusive of such combinations are generally semi-solid in nature.

As used herein, “LCST” or “lower critical solution temperature,” refers to the temperature at which the pentablock polymer undergoes reverse thermal gelation, i.e., the temperature below which the polymer is soluble in water and above which the pentablock polymer undergoes phase separation to form a semi-solid containing the drug and the pentablock polymer. The terms “LCST,” “gelation temperature,” and “reverse thermal gelation temperature,” or the like shall be used interchangeably in referring to the LCST.

As used herein, “hydrophilic” refers to the ability to dissolve in water. When used in the context of the hydrophilic drugs or diagnostic agents in the present disclosure, the term embraces a drug that is preferably sparingly soluble, more preferably soluble, still more preferably freely soluble, and still most preferably very soluble, according to USP-NF definitions.

As used herein, “parenteral” shall mean any route of administration other than the alimentary canal and shall specifically include intramuscular, intraperitoneal, intra-abdominal, intra-articular, subcutaneous, and, to the extent feasible, intravenous.

As used herein, “pharmaceutically acceptable” shall refer to that which is useful in preparing a pharmaceutical composition that is generally safe, non-toxic, and neither biologically nor otherwise undesirable and includes that which is acceptable for veterinary use as well as human pharmaceutical use. Examples of “pharmaceutically acceptable liquid carriers” include water and organic solvents. Preferred pharmaceutically acceptable aqueous liquids include PBS, saline, and dextrose solutions.

As used herein, “peptide”, “polypeptide”, “oligopeptide,” and “protein” shall be used interchangeably when referring to peptide or protein drugs and shall not be limited as to any particular molecular weight, peptide sequence or length, field of bioactivity, diagnostic use, or therapeutic use unless specifically stated.

As used herein, “solution,” “aqueous solution,” and the like, when used in reference to a combination of drug and pentablock polymer contained in such solution, shall mean a liquid-based solution having such drug/polymer combination dissolved or substantially uniformly suspended therein at a functional concentration and maintained at a temperature below the LCST of the block polymer.

As used herein, “thermosensitive” refers to a polymer which exists as a generally clear dispersion near ambient temperature in water but when the temperature is raised the LCST (which is preferably about body temperature), interact to form a gel.

The term “treatment” or “treating” means administration of a drug for purposes including: (i) preventing the disease or condition, that is, causing the clinical symptoms of the disease or condition not to develop; (ii) inhibiting the disease or condition, that is, arresting the development of clinical symptoms; and/or (iii) relieving the disease or condition, that is, causing the regression of clinical symptoms.

Below, the exemplary embodiments are shown and specific language will be used herein to describe the same. It should nevertheless be understood that no limitation of the scope of the disclosure is thereby intended. Alterations and further modifications of the inventive features illustrated herein, and additional applications of the principles of the present disclosure as illustrated herein, for one skilled in the relevant art, in connection with this disclosure, should be considered within the scope of the present disclosure.

### Biodegradable Thermosensitive Pentablock Polymers

The present disclosure is directed to pentablock polymers comprised of (A) PLA, (B) PCL, (C) PEG, and/or (D) PGA. Generally, the block polymer will be a pentablock polymer, i.e., a CBABC, denoted as a “PEG terminal” arrangement or DBCBD type block polymer, denoted as a “PEG central” arrangement.

For preparation of the pentablock polymer used for the thermosensitive gels of the present disclosure, in some embodiments, the pentablock polymer preferably has a PEG-PCL-PLA-PCL-PEG, “PEG terminal” configuration. In some embodiments, the pentablock polymer preferably has a PGA-PCL-PEG-PCL-PGA, “PEG central” configuration.

### PEG Terminal Composition

For preparation of the pentablock polymer of the present disclosure, the pentablock polymer can have a “PEG Terminal” block configuration, comprising CBABC.

The hydrophobic A block segment is preferably derived from a lactide. The A block segment preferably comprises PLA having an average molecular weight of between about 100 to 5,000 Da, more preferably between about 150 and 1,500 Da, and still more preferably between about 250 and 1100 Da (for example, an average molecular weight of about 250 Da, 300 Da, 400 Da, 500 Da, 600 Da, 700 Da, 800 Da, 1000 Da, 1100 Da or some range there between). An average molecular weight in the range of about 250 to 1100 Da is most preferred. It will be appreciated that in the preferred embodiment, a linker separates the hydrophobic A block, but that the average molecular weight referenced for this block refers to the combined molecular weights of the PLA blocks on both sides of the linker.

The hydrophobic B block segment is preferably derived from a cyclic lactone, and is most preferably derived from ε-caprolactone. Thus, in one aspect, the B block segment comprises PCL having an average molecular weight less than about 3000 Da. For example, the B block segment is preferably PCL having an average molecular weight of between about 100 to 3000 Da (for example, and average molecular weight of about 200 Da, 300 Da, 400 Da, 500 Da, 600 Da, 700 Da, 800 Da, 900 Da, 1000 Da, 1100 Da, 1200 Da, 1300 Da, 1400 Da, 1500 Da, 1600 Da, 1700 Da, 1800 Da, 1900 Da, 2000 Da, 2100 Da, 2200 Da, 2300 Da, 2400 Da, 2500 Da, 2600 Da, 2700 Da, 2800 Da, 2900 Da, 3000 Da, or some range therebetween), and more preferably has an average molecular weight between about 200 and 2000 Da, and still more preferably has an average molecular weight between about 400 to 1500 Da.

The hydrophilic C block segment is preferably PEG having an average molecular weight of between about 100 to 1000 Da and more preferably has an average molecular weight between about 350 to 750 Da, and still more preferably has an average molecular weight between about 400 to 550 Da.

Thus, in one aspect, a pentablock polymers used to make the “PEG terminal” thermosensitive gel in accordance with the present disclosure may be defined according to the following formula:

PEG_{C}-PCL_{B}-PLA_{A}-PCL_{B}-PEG_{C}

wherein A defines an average molecular weight of about 100 to about 5,000 Da, preferably about 150 to about 1,500 Da, more preferably about 250 to about 1,100 Da;
wherein B defines an average molecular weight of 100 to about 3000 Da, preferably about 200 to about 2000 Da, more preferably about 400 to about 1500 Da; and
wherein C defines an average molecular weight of about 100 to about 1000 Da, preferably about 350 to about 750 Da, more preferably about 400 to about 550 Da.

In some embodiments, the total molecular weight for the polymer can be about 1500-10000 Da, preferably about 2000-7000 Da, and more preferably about 2500-5000 Da.

A linker, such as diisocyanate, for example 1,4-diisocyanatebutate, 1,4-diisocyante phenylene, or hexamethylene diisocyanate can be included in the PEG terminal polymer.

By varying the molecular weights of the various A, B, and C blocks, the pentablock polymers synthesized as disclosed herein have various hydrophobic and hydrophilic blocks, which affect the release rate and duration of release of active agents, Further, the hydrophilic C block and the hydrophobic A and B blocks are synthesized and utilized because of their unique interactions with hydrophilic and hydrophilic active agents. Generally, for the preparation of extended release compositions, the hydrophilic C block (PEG block) should be less than 50% by weight, the B block (PCL block) should be greater than 10% by weight, and the A block (PLA block) should be less than 50% by weight.

In various embodiments, the pentablock polymers disclosed herein provide the surprising characteristic of sustained release of various drugs, irrespective of the drug's hydrophobic or hydrophilic nature or molecular weight. Indeed, it has been surprisingly discovered that by increasing hydrophobicity of the pentablock polymer, by increasing PCL, PLA and/or PGA concentrations, and/or by decreasing PEG concentration, more prolonged, sustained release of drugs can be achieved.

The molecular weight of the hydrophobic A and B blocks, relative to that of the water-soluble C block, is regulated to be sufficiently small to retain desirable water-solubility and gelling properties. In addition, for the preparation of gels, the proportionate weight ratios of hydrophilic C block to the more hydrophobic A and B blocks must also be sufficient to enable the block polymer to possess water solubility at temperatures below the LCST.

As shown in the following examples, the pentablock polymer compounds of the present disclosure are ideally suited to form composition, which may include an effective amount of active agents, such as biologics or small molecules. In general, the pentablock polymer can be designed to have a selected rate of drug release, and typically drug release. However, the drug and/or diagnostic agent typically comprises about 0.01 to 50 wt % of the composition, more preferably about 0.1 to 30% wt of the composition, with about 1 to 10 wt % being most preferred.

The desired hydrophobicity for tunable drug release can also be achieved by admixing two or more pentablock co-polymers in various ratios. As an example, PTS 10GH and PTS 17GH were mixed in 1:1 ratio and were tested for in vivo release in mice (see Examples).

### PEG Central Composition

In some embodiments, the pentablock polymer preferably has a PGA-PCL-PEG-PCL-PGA configuration, denoted “PEG central.”

For preparation of the pentablock polymer of the present disclosure, the pentablock polymer can have a “PEG Central” block configuration, comprising DBCBD.

The hydrophobic D block segment is preferably derived from a glycolide. The D block segment preferably comprises PGA having an average molecular weight of between about 100 to 5,000 Da, still more preferably between about 150 to 1,500 Da, still more preferably between about 250 and 1100 Da (for example, the D block segment may have an average molecular weight of about 200 Da, 300 Da, 400 Da, 500 Da, 600 Da, 700 Da, 800 Da, 900 Da, 1000 Da, 1100 Da or some range therebetween).

The hydrophobic B block segment is preferably derived from a cyclic lactone, and is most preferably derived from ε-caprolactone. Thus, the B block segment comprises PCL having an average molecular weight of between about 100 Da to 3000 Da, more preferably between about 200 to 2000 Da, still more preferably about 400 to 1500 Da.

The hydrophilic C block segment is preferably PEG having an average molecular weight of between about 100 to 1000 Da and more preferably has an average molecular weight between about 350 and 750 Da, and still more preferably has an average molecular weight between about 400 to 550 Da, and most preferably has an average molecular weight of less than about 550 Da.

In some embodiments, different forms of PEG can be used, depending on the initiator used for the polymerization process. For example, the PEG can be methyl ether PEG (m-PEG). Different molecular weight (MW) combinations of m-PEG as a starting point can also be used. For example, the m-PEG can be a combination of two or more m-PEG having different MW ranging from 100-10000 Da, e.g., MW_{X}+MW_{Y} such as MW 400+MW 550 at a 1:1 ratio or any other ratio. The polymers can also be combined after synthesis with m-PEG MWx and m-PEG MW_{Y} separately.

Thus, in one aspect, a pentablock polymers used to make an extended release polymer in accordance with the present disclosure may be defined according to the following formula:

PGA_{D}-PCL_{B}-PEG_{C}-PCL_{B}-PGA_{D}

wherein D defines an average molecular weight of 100 to about 5,000 Da, preferably about 150 to about 1,500 Da, more preferably about 250 to about 1,100 Da;
wherein B defines an average molecular weight of about 100 to about 3000 Da, preferably about 200 to about 2000 Da, more preferably about 400 to about 1500 Da; and
wherein C defines an average molecular weight of about 100 to about 1000 Da, about 350 to about 750 Da, preferably about 400 to about 550 Da.

In some embodiments, the total molecular weight for the polymer can be about 1500-10000 Da, preferably about 2000-7000 Da, and more preferably about 2500-5000 Da.

By varying the molecular weights of the various B, C, and D blocks, the pentablock polymers synthesized as disclosed herein have various hydrophobic and hydrophilic blocks, which affect the release rate and duration of release of active agents. Further, the hydrophilic C block and the hydrophobic B and D blocks are synthesized and utilized because of their unique interactions with hydrophobic and hydrophilic active agents. Both hydrophilic and hydrophobic drugs are expected to be sustained more by the more hydrophobic polymers. Generally, for the preparation of extended release compositions, the hydrophilic C block (PEG block) should be less than 50% by weight, the B block (PCL block) should be greater than 10% by weight, and the D block (PGA block) should be less than 50% by weight.

The molecular weight of the hydrophobic B and D blocks, relative to that of the water-soluble C block, is regulated to be sufficiently small to retain desirable water-solubility and gelling properties. In addition, for the preparation of gels, the proportionate weight ratios of hydrophilic C block to the more hydrophobic B and D blocks must also be sufficient to enable the block polymer to possess water solubility at temperatures below the LCST.

As shown in the following examples, the pentablock polymer compounds of the present disclosure are ideally suited to form composition, which may include an effective amount of active agents, such as biologics or small molecules. In general, the pentablock polymer can be designed to have a selected rate of drug release, and typically drug release. However, the drug and/or diagnostic agent typically comprises about 0.01 to 50 wt %) of the composition, more preferably about 0.1 to 20 wt % of the composition, with about 1 to 10 wt % being most preferred.

### Pentablock Polymer Properties

The mixture of the pentablock polymer used for thermosensitive gels and the bioactive agent or diagnostic agent may be prepared as an aqueous dispersion at a lower temperature than the gelation temperature of the pentablock polymer. In general, this may be performed by forming a dispersion of the pentablock polymer and the bioactive agent or diagnostic agent at a suitable temperature. The pentablock polymers are generally in solution at room temperature (typically about 20 to 26° C.) or at the desired storage temperature (e.g., refrigeration). Once parenterally injected into the body, e.g., via intramuscular, subcutaneous, intraperitoneal, intrathecal, intra-articular (e.g., knee injection), intraocular or topical route as a drug delivery liquid, the drug/polymer formulation will undergo a phase change and will preferably form a firm or solid gel since the body temperature (e.g., 37° C. for humans) will be above the gelation temperature of the material (typically about 30 to 35° C.). The LCST is thus preferably less than about 35, 34, 33, 32, 31, or 30° C. That is, the composition comprising the pentablock polymer forms a gel and solidifies into a depot as the temperature is raised due to the reverse gelation properties of the drug/polymer composition.

The pentablock polymer and bioactive agent or diagnostic agent system will cause minimal toxicity and mechanical irritation to the surrounding tissue due to the biocompatibility of the materials and will be completely biodegradable within a specific predetermined time interval. Once gelled, the release of the bioactive agent or diagnostic agent from the polymeric matrix can be controlled by proper formulation of the various polymer blocks.

The concentration at which the pentablock polymers are soluble at temperatures below the LCST may be considered as the functional concentration. Generally speaking, polymer concentrations of up to about 50% by weight can be used and still be functional. However, concentrations in the range of about 3 to 40% are preferred and concentrations in the range of about 10 to 25% by weight are most preferred. In order to obtain a viable phase transition of the polymer, a certain minimum concentration is required. At the lower functional concentration ranges the phase transition may result in the formation of an emulsion rather than a gel. At higher concentrations, a gel network is formed. The actual concentration at which an emulsion may phase into a gel network may vary according to the ratio of hydrophobic A and B blocks to hydrophilic C blocks and the composition and molecular weights of each of the blocks. Since both emulsions and gels can both be functional it is not imperative that the actual physical state be precisely determined. However, the formation of a swollen gel network is preferred.

In various embodiments, the pentablock polymers disclosed herein provide the surprising characteristic of sustained release of various drugs, irrespective of the drug's hydrophobic or hydrophilic nature or molecular weight. Indeed, it has been surprisingly discovered that by increasing hydrophobicity of the pentablock polymer, by increasing PCL, PLA and/or PGA concentrations, and/or by decreasing PEG concentration, more prolonged, sustained release of drugs can be achieved.

The desired hydrophobicity for tunable drug release can also be achieved by admixing two or more pentablock co-polymers in various ratios. As an example, PTS 10GH and PTS 17GH were mixed in 1:1 ratio and were tested for in vivo release in mice.

### Pentablock Polymer Applications

The biodegradable thermosensitive gels comprising the pentablock polymers of the present disclosure provide for controlled or extended release of a hydrophilic or hydrophobic agent, such as a biologic or small molecule drug. In general, the pentablock polymer can be designed to have a selected rate of drug release.

The biodegradable, thermosensitive pentablock polymers of the current disclosure can be formulated as pharmaceutical compositions, to be administered to a mammalian host, such as a human patient in a variety of forms, such as a gel formulation. Suitable forms of polymer administration can include injection or administration methods to regions such as: intradermal, subcutaneous, intramuscular, intravitreal, intraocular, intraarticular, intracardiac, intralesional, intraperitoneal, intracerebroventricular, intrathecal, intraosseous infusion, intracerebral, intrauterine, intravaginal, extraamniotic, intracavernous, and/or intravesica. The polymers of the present disclosure can be used as vitreous body substitutes, viscoelastic surgical gels, for example, for use in cataract surgery, retinal detachment surgery, and the like, as well as for ear treatments and oral treatments (e.g., dry mouth treatment).

The polymers can be administered by injection, in pure liquid form, or as suspensions. The polymers can be prepared in water, buffer solution, or optionally mixed with nontoxic surfactants. Under ordinary conditions of storage and use, these preparations contain a preservative to prevent the growth of microorganisms.

The pentablock polymers of the present disclosure are used to form biodegradable thermosensitive gels are generally used in depot drug delivery, the pentablock polymers can also be used in a variety of therapeutic applications or diagnostic applications.

Therapeutic applications that can benefit from the use of pentablock polymers as a delivery vehicle can include, but not limited to, the treatment of various conditions in need of extended release therapeutics and/or a gel composition. For example, the polymer composition of the present disclosure can be utilized to deliver therapeutics for the treatment of: age related disorders (e.g., bone decalcification, menopause, joint degradation), cardiac disorders (e.g., atrial fibrillation), cancer treatment (i.e. chemotherapy, targeted cancer cell treatments), dermatological preparations and/or disorders (e.g., acne, dermal rashes or infections), immunosuppressants (e.g., tissue transplants, immune disorders), metabolic conditions (e.g., diabetes, obesity), muscular-skeletal conditions (e.g., anabolic/catabolic tissue stimulation, pain management, regeneration of tissue), oral treatments (e.g., dry mouth treatments, delivery of analgesics, antibiotics, or other agents), pain management (e.g., acute, chronic, or intermediate duration pain symptoms), psychiatric disorders (e.g., schizophrenia, bi-polar disorder, major depressive disorder), ophthalmic disorders (e.g., glaucoma, macular degeneration) and arthritis.

Exemplary therapeutics that can benefit from the use of pentablock polymers as the polymer composition of the present disclosure can include various hydrophobic drugs, hydrophilic drugs, or combinations of hydrophobic and hydrophilic drugs. For example, the polymer composition can be utilized to deliver therapeutic such as, biologics and small molecule drugs, including but not limited to: angiogenesis inhibitors (e.g., pazopanib), antibiotics (e.g., penicillins, cephalosporins, carbapenems, macrolides, aminoglycosides, quinolones (i.e., fluoroquinolones), sulfonamides, tetracyclines), anti-inflammatories (e.g., nonsteroid antiinflamatory drugs (NSAIDS) (i.e. celecoxib), cyclooxygenase (COX) inhibitors (i.e. naproxen, difluprednate), Beta-blockers (e.g., propranolol), calcium channel blockers (e.g., verapamil), chemotherapeutics (e.g., tyrosine-kinase inhibitors (i.e., gleevec), cytotoxic antibiotics—(i.e., bleomycin), topoisomerase inhibitors (i.e., topotecan), hormones (e.g., estrogen, testosterone, human growth hormone, prolactin), immunosuppressants (e.g., cyclosporine), metabolic regulatory modalities (e.g., insulin), pain medications (e.g., narcotics, NSAIDS, opioids), psychiatric drugs (e.g., antidepressants, antipsychotics, mood stabilizers), ophthalmic medications (e.g., carbonic anhydrase inhibitors—brinzolamide), steroids (i.e., progestogens—progesterone, corticosteroids, mineralocorticoids—aldosterone, glucocorticoids—cortisol, androgens—testosterone, estrogens—estrogen), stem cells (e.g., burn wound healing, cancer therapy), gene therapies, delivery of viral vectors (e.g., construct delivery methods).

### EXAMPLES

### Example 1—Synthesis of Thermosensitive Biodegradable Pentablock Co-Polymer

In this example, a pentablock polymer having a PEG-PCL-PLA-PCL-PEG block configuration was prepared.

For synthesis a polyethylene glycol-polycaprolactone (PEG-PCL) diblock copolymer was synthesized by ring opening polymerization of ε-caprolactone with monomethoxy polyethylene glycol (mPEG) using tin octoate as a catalyst. First, a predetermined amount of mPEG 550 and ε-caprolactone were added in a round bottom flask equipped with a stir bar. Polymer was vacuum purged four times with nitrogen, followed by addition of 0.5 wt % of mPEG and ε-caprolactone combined of tin octoate catalyst. The reaction mixture was heated to 130° C. for 36 hours under nitrogen (Step 1). Next, the resulting diblock copolymer was re-heated to 130° C. and L-lactide was added. The reaction mixture was vacuum purged four times with nitrogen followed by addition of 0.5 wt % of entire DB and lactide combined of tin octoate catalyst and the reaction mixture was heated to 130° C. for 36 hours under nitrogen (Step 2).

The resulting triblock polymer was then dissolved in dichloromethane and precipitated by addition of chilled heptanes (−78° C.). The heptane was then decanted and the precipitate was vacuum-dried to remove any residual solvents.

Then, the resulting triblock copolymer was coupled utilizing hexamethylenediisocyanate (HMDI) as a linker to prepare PEG-PCL-PLA-PCL-PEG pentablock copolymers. Coupling reaction was carried out at 80° C. for 8 hours (step 3). The resulting polymer is re-purified by precipitation and tin is scavenged. The purified pentablock is stored at −20° C. Synthesis of PEG-PCL-PLA-PCL-PEG block configuration is:

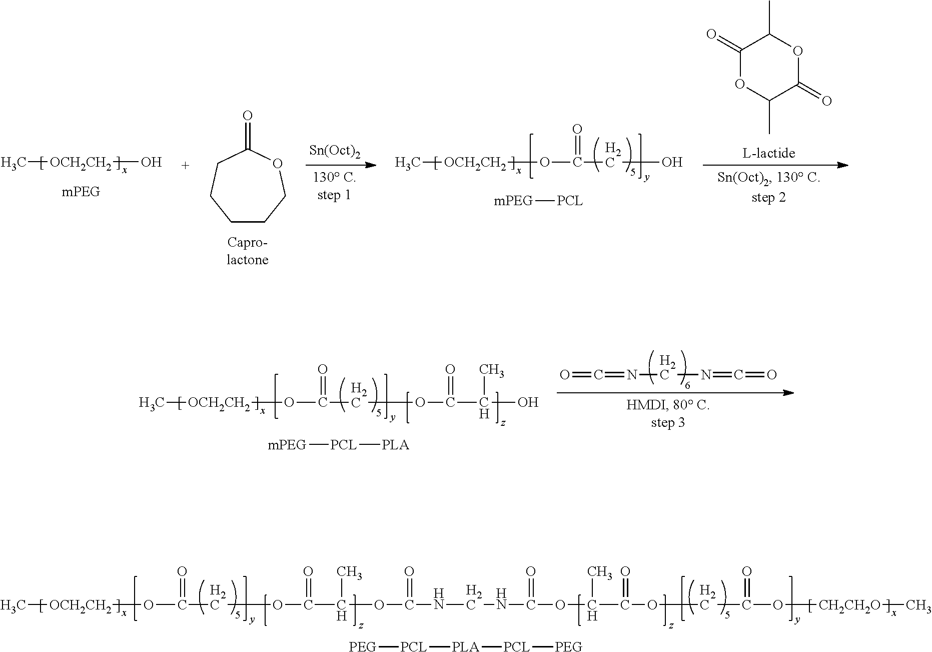

### Example 2—Synthesis of Thermosensitive Biodegradable Pentablock Co-Polymers

In this example, various embodiments of themosensitive biodegradable pentablock co-polymers were synthesized. Monomethoxy PEG (550), L-lactide, tin octoate, hexamethylenediisocynate (HMDI), sodium sulfate, dichloromethane, heptanes, endotoxin free water were purchased from Sigma-Aldrich (St. Louis, Mo.). ε-Caprolactone was purchased from Alfa Aesar (Ward Hill, Mass.). PTSgels with PEG-PCL-PLA-PCL-PEG block arrangements were synthesized as previously described by Patel et al. (Novel Thermosensitive Pentablock Copolymers for Sustained Delivery of Proteins in the Treatment of Posterior Segments Diseases, (2014) pp 1185-1200) and Patel et al. (Tailor-made pentablock copolymer based formulation for sustained ocular delivery of protein therapeutics, Invest. Ophthalmol. Vis. Sci. 55 (2014) p. 4629 and as described in Example 1). Briefly, the diblock copolymer was synthesized by ring-opening copolymerization of ε-caprolactone with monomethoxy PEG using tin octoate as a catalyst. The resulting diblock copolymer was similarly converted to triblock by adding L-lactide. The resulting triblock copolymer was coupled utilizing hexamethylenediisocyanate (HMDI) as a linker to prepare PEG-PCL-PLA-PCL-PEG pentablock copolymers. The purified pentablock is stored at −20° C., until used.

Several exemplary biodegradable thermosensitive PTSgel polymers were synthesized:
i. (PBC-10GH) PEG₅₅₀-PCL₅₀₀-PLA₁₀₀₀-PCL₅₀₀-PEG₅₅₀(Mw=3100, PEG=35.5%)
ii. (PBC-101GH) PEG₅₅₀-PCL₄₀₀-PLA₁₁₀₀-PCL₄₀₀-PEG₅₅₀(Mw=3000, PEG=36.7%)
iii. (PBC-102GH) PEG₅₅₀-PCL₅₅₀-PLA₁₁₀₀-PCL₅₅₀-PEG₅₅₀(Mw=3300, PEG=33.3%)
iv. (PBC-103GH) PEG₅₅₀-PCL₇₀₀-PLA₁₁₀₀-PCL₇₀₀-PEG₅₅₀(Mw=3600, PEG=30.5%)
v. (PBC-112GH) PEG₅₀₀-PCL₅₅₀-PLA₁₁₀₀-PCL₅₅₀-PEG₅₀₀(Mw=3200, PEG=31.3%)
vi. (PBC-113GH) PEG₅₀₀-PCL₇₀₀-PLA₁₁₀₀-PCL₇₀₀-PEG₅₀₀(Mw=3500, PEG=28.6%)
vii. (PBC-114GH) PEG₅₀₀-PCL₉₀₀-PLA₁₁₀₀-PCL₉₀₀-PEG₅₀₀(Mw=3900, PEG=25.6%)
viii. (PBC-17GH) PEG₅₅₀-PCL₁₂₅₀-PLA₁₁₀₀-PCL₁₂₅₀-PEG₅₅₀(Mw=4700, PEG=23.4%)
ix. (PBC-119GH) PEG₄₀₀-PCL₅₀₀-PLA₁₀₀₀-PCL₅₀₀-PEG₄₀₀(Mw=2800, PEG=28.5%)
x. (PBC-122GH) PEG₅₀₀-PCL₈₀₀-PLA₁₁₀₀-PCL₈₀₀-PEG₅₀₀(Mw=3700, PEG=27.0%)
xi. (PTS-203GH) PEG₅₀₀-PCL₁₁₀₀-PLA₁₁₀₀-PCL₁₁₀₀-PEG₅₀₀(MW=4300, PEG=23.3%)
xii. (PTS-204GH) PEG₅₀₀-PCL₁₂₅₀-PLA₁₁₀₀-PCL₁₂₅₀-PEG₅₀₀(MW=4600, PEG=21.7%)
xiii. (PTS-205GH) PEG₅₀₀-PCL₉₀₀-PLA₁₁₀₀-PCL₉₀₀-PEG₅₀₀(MW=3900, PEG=25.6%)
xiv. (PTS-206GH) PEG₅₀₀-PCL₁₀₀₀-PLA₁₁₀₀-PCL₁₀₀₀-PEG₅₀₀(MW=4100, PEG=24.4%)
xv. (PTS-209GH) PEG₄₀₀-PCL₇₀₀-PLA₁₁₀₀-PCL₇₀₀-PEG₄₀₀(MW=3300, PEG=24.2%)
xvi. (PTS-210GH) PEG₄₀₀-PCL₉₀₀-PLA₁₁₀₀-PCL₉₀₀-PEG₄₀₀(MW=3700, PEG=21.6%)
xvii. (PTS-211GH) PEG₄₀₀-PCL₅₀₀-PLA₁₁₀₀-PCL₅₀₀-PEG₄₀₀(MW=2900, PEG=27.6%)
xviii. (PTS-212GH) PEG₄₀₀-PCL₆₀₀-PLA₁₁₀₀-PCL₆₀₀-PEG₄₀₀(MW=3100, PEG=25.8%)
xix. (PTS-214GH) PEG₅₅₀-PCL₁₀₀₀-PLA₂₀₀₀-PCL₁₀₀₀-PEG₅₅₀(MW=5100, PEG=21.6%)
xx. (PTS-216GH) PEG₄₀₀-PCL₄₀₀-PLA₁₁₀₀-PCL₄₀₀-PEG₄₀₀(MW=2700, PEG=29.6%)
xxi. (PTS-217GH) PEG₄₀₀-PCL₃₀₀-PLA₁₁₀₀-PCL₃₀₀-PEG₄₀₀(MW=2500, PEG=32%)
xxii. (PTS-303GH) PEG₄₇₅₍₄₀₀₊₅₅₀₎-PCL₉₀₀-PLA₁₁₀₀-PCL₉₀₀-PEG₄₇₅₍₄₀₀₊₅₅₀₎(MW=3850, PEG=24.7%)

The polymers were constructed with different block sizes of m-PEG, PCL and PLA with PLA in the center of the molecule (m-PEGx-PCLy-PLAz-PCLy-PEGx-m). The molecular weight in the provided examples ranged between 2,500-4,700 Da with gradual increase in the hydrophobicity of molecules. The objective was to vary molecular weights and hydrophobic-hydrophilic block ratios in the polymers to achieve modulation of drug release. Polymers were characterized by NMR, FTIR for structural confirmation, by GPC for PDI determination and ability to transition from liquid phase to gel at 37° C. and by DLS for particle size determination in aqueous dispersion. Several polymers were compared for in vitro release profiles. Two polymers 10GH and 113 GH were used for in vivo subcutaneous release, polymer disappearance and safety investigations. 102GH and 10GH at various concentrations was also analyzed for in vitro degradation analyses.

### Example 3—Validation of Synthesized Polymer Composition

In this example, Fourier-Transform Infrared Spectroscopy (FTIR), ¹H NMR and Gel Permeation Chromatography (GPC) spectroscopy analysis was performed to characterize the polymer.

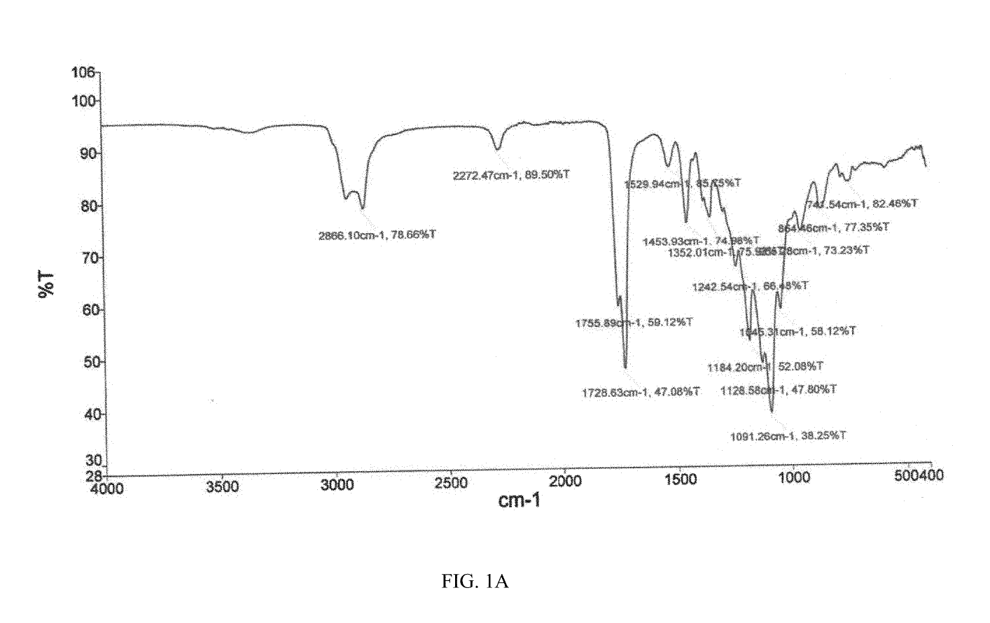

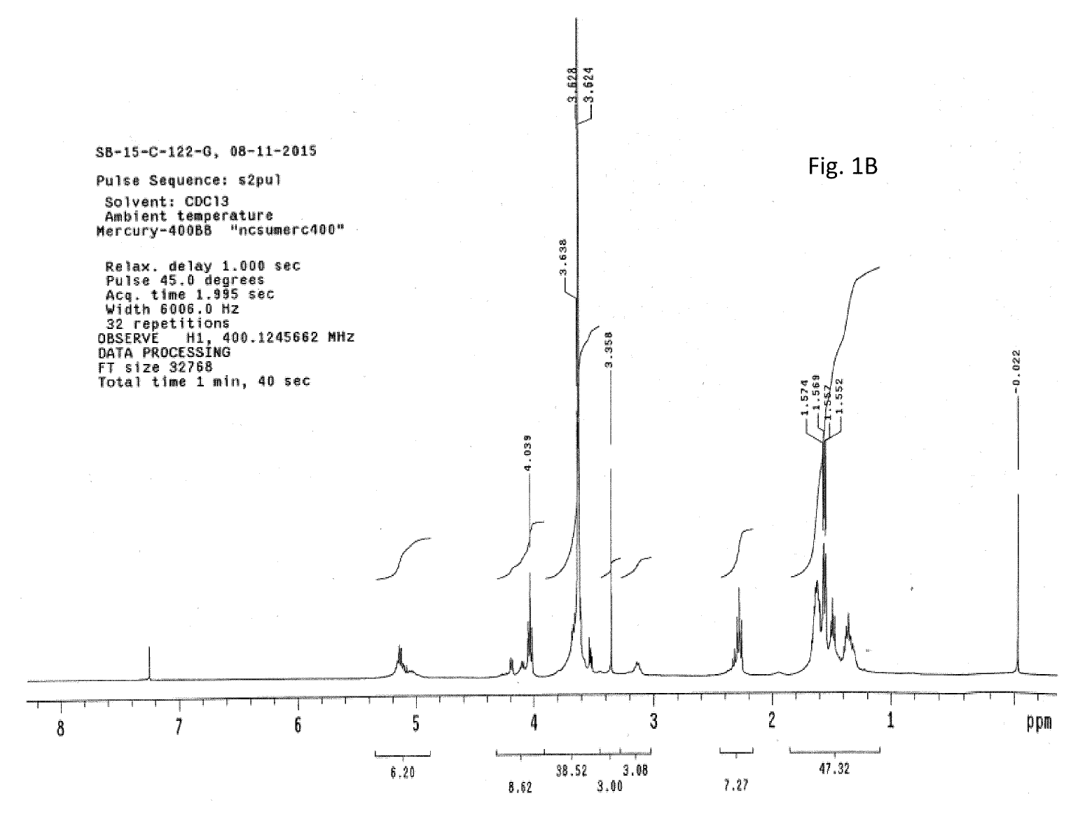

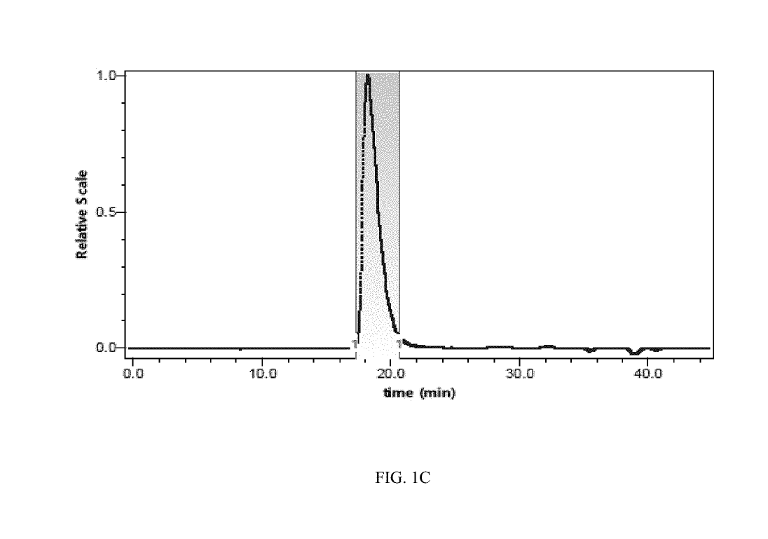

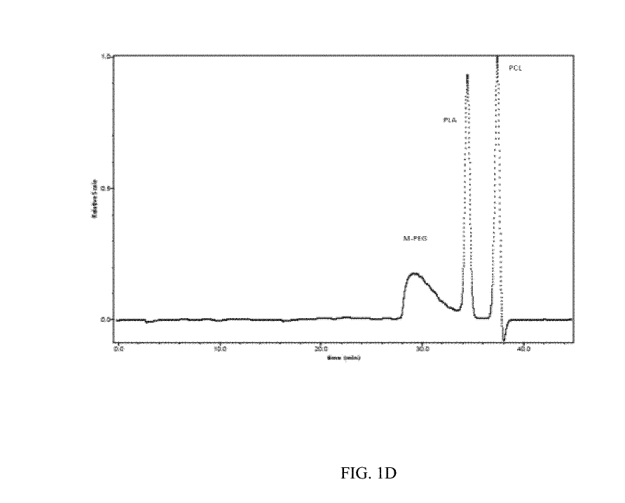

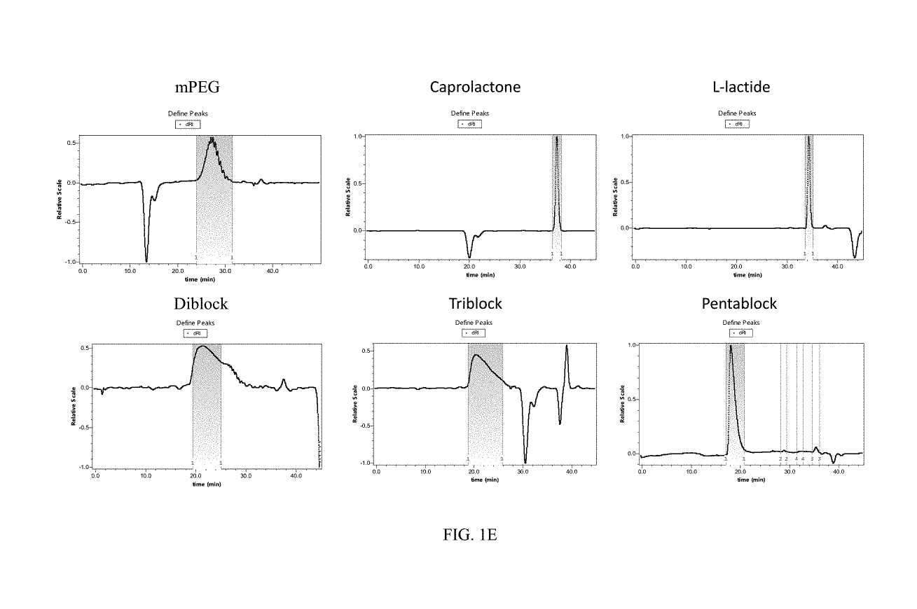

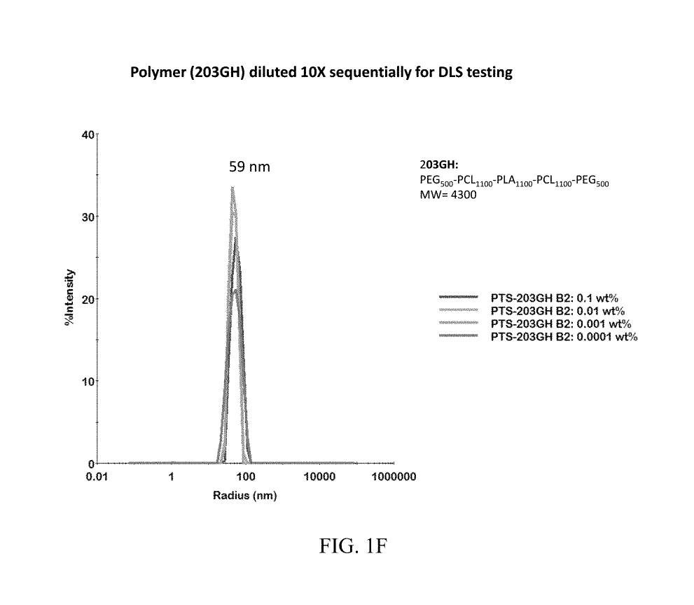

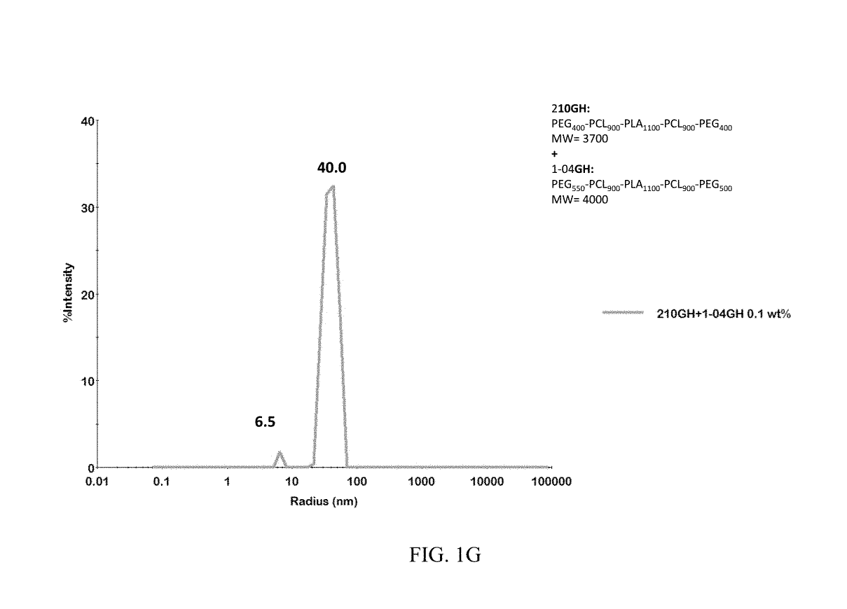

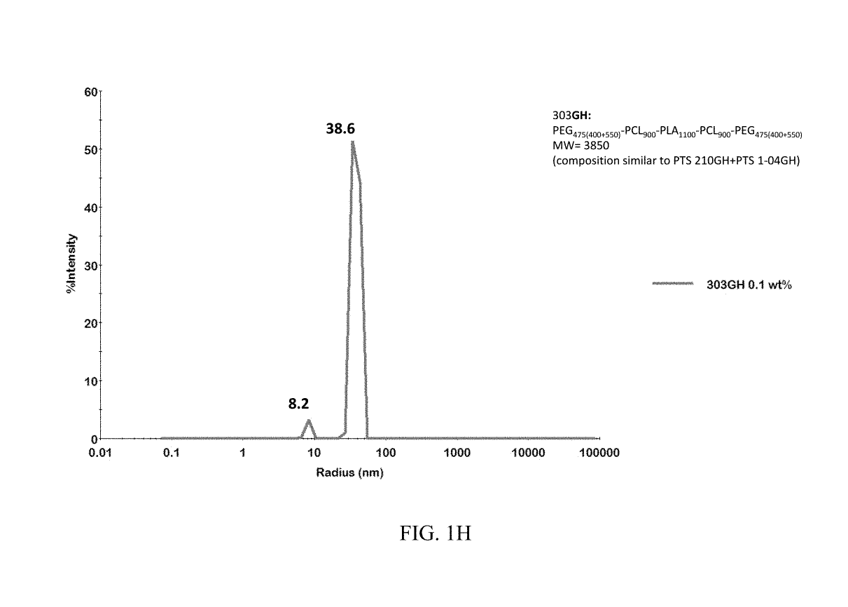

FITR spectra were recorded with a Perkin Elmer Spectrum Version 10.03.09 infrared spectrophotometer. FTIR scan of neat polymer was carried out in a range of 4000-400 cm-1. The results for FTIR spectrum analysis of 10GH polymer is shown in FIG. 1A. An absorption band at 1729 cm-1 and multiple bands ranging 1000-1300 cm-1 established the presence of ester linkages in pentablock co-polymer. Existence of terminal hydroxyl group was confirmed by C—O stretching band at 1089 cm-1 and O—H band (stretch) in the range of 3300-3400 cm-1. C—H stretching bands at 2938 and 2866 cm-1 depicted presence of PCL blocks. Absorption band at 1531 cm-1 (N—H stretching) exhibited the formation of urethane group in pentablock co-polymer.

¹H NMR spectroscopy was performed to characterize the polymer composition. Molecular structure and molecular weight (Mn) of the PTSgel were analyzed utilizing a Mercury 300-MHz NMR spectrometer. 1H-NMR spectrograms were recorded by dissolving the polymers in deuterated chloroform (CDCl3).

The results for ¹H NMR spectroscopy analysis of 10GH polymer is shown in FIG. 1B. Typical ¹H-NMR characteristic peaks were observed at 1.55, 2.30 and 4.04 δ ppm representing methylene protons of —(CH₂)₃—, —OCOCH₂—, and —CH₂OOC— of PCL units, respectively. A sharp peak at 3.64 δ ppm was attributed to methylene protons (—CH₂CH₂O—) of PEG. Typical signals at 1.50 (—CH₃) and 5.17 (—CH—) δ ppm were assigned for PLA blocks. Whereas, a peak at 3.36 δ ppm was denoted to terminal methyl of (—OCH3-) of PEG. The [EO-[CL]-[LA] molar ratios of final products were calculated from integrations of PEG signal at 3.64 δ ppm, PCL signal at 4.04 δ ppm and PLA signal at 5.17 δ ppm. PEG signal at 3.64 δ ppm was applied for the calculation of molar ratio of various blocks within the pentablock co-polymer. Referring to Table 1, the estimated molecular weight, calculated using NMR, was reported to be close to the theoretical feed ratio.

Gel Permeation Chromatography (GPC) analysis was performed to characterize the polymer. Molecular weights (Mn and Mw) and polydispersity of polymers were examined by GPC analysis. Briefly, 20 mg of polymer was dissolved in 1 mL of tetrahydrofuran (THF). Polymer samples were separated on two oligopore columns (Agilent, Santa Clara, Calif.) connected in series and maintained at 40° C. Solvent THF at the rate of 0.5 mL/min was utilized as eluting solvent. Samples were analyzed on Wyatt technologies MINI DAWN instrument (S. No. 528-T) connected to OPTILAB DSP interferometric refractometer, using ASTRA 6 software.

A Typical GPC chromatogram 10GH pentablock copolymer is shown in FIG. 1C. Molecular weight (Mw and Mn) and polydispersity of polymers were determined by GPC. A single peak for the polymer was observed describing unimodal distribution of molecular weight and absence of any other homopolymer block such as PEG, PCL or PLA. Polydispersity (PDI) for the five analyzed polymers ranged from 1.08-1.28 indicating narrow distribution of molecular weights. Estimated molecular weights of synthesized PTSgel were close to the feed ratio (Table 1). GPC chromatogram for reference standards (m-PEG, PCL and PLA) are shown in FIG. 1D. GPC analysis using an Oligopore column (Agilent) for various pentablock components are shown in FIG. 1E.

TABLE 1
MW, Mn and PDI determination of PTSgels

| PTSgel | Total Mn^{a} | Total Mn^{b} | Total Mn^{c} | Mw^{c} | |
|---|---|---|---|---|---|
| ID | (theoretical) | (calculated) | (calculated) | (GPC) | PDI^{c} |
| 101GH | 3000 | 4251 | 4784 | 5132 | 1.07 |
| 10GH | 3100 | 3513 | 4855 | 5264 | 1.08 |
| 103GH | 3600 | 3896 | 3404 | 4347 | 1.28 |
| 113GH | 3500 | 4088 | 4615 | 5078 | 1.1 |
| 122GH | 3700 | 4433 | 4349 | 4941 | 1.14 |

^{a}Theoretical value, calculated according to the feed ratio
^{b}Calculated from ¹H NMR
^{c}Determined by GPC analysis.

### Example 4—Preparation and Size Characterization of Pentablock Copolymers Using Dynamic Light Scattering (DLS)

A pentablock copolymer solution was dissolved at 11 mg/mL in HPLC pure water and stored at 4° C. until analyzed. These solutions were analyzed as is or after further dilution for their size using dynamic light scattering (DLS) with a Wyatt 233-MOB Mobius instrument (Mw-R model: Globular proteins). The analysis was performed at an angle of 163.5° at 20° C. For each sample, the mean radii were obtained after five runs of ten acquisitions.

FIG. 1F shows DLS spectrum for a polymer (PTS 203GH) after up to 100× dilution of 11 mg/mL sample. Particle size remained unchanged and there is one peak observed in the spectrum.

FIG. 1G shows a DLS spectrum of two polymers combined (PTS 210+PTS1-04). By mixing the polymer of different composition should help provide more flexibility in achieving drug release modulation.

FIG. 1H shows a similar outcome on DLS analysis when a polymer synthesis (PTS 303GH) itself was initiated by combining m-PEG of different sizes. Polymer in FIG. 1H is theoretically similar to what was generated by mixing two polymers synthesized separately as shown in FIG. 1G.

### Example 5—Solution-Gel Transition Studies of Polymer Compositions

In this example, the sol (flow)-gel (no flow) transition of PTSgel were examined. Briefly, the polymers were dissolved in PBS buffer (pH 7.4) at 25 wt % concentration A 0.5 mL of aqueous polymeric solution was transferred into 2.5 mL glass vial and placed in water bath maintained at 37° C. Vials were kept for 5 min at 37° C. Gel formation was observed visually by inverting the tubes, immediately after pulling out of the water bath.

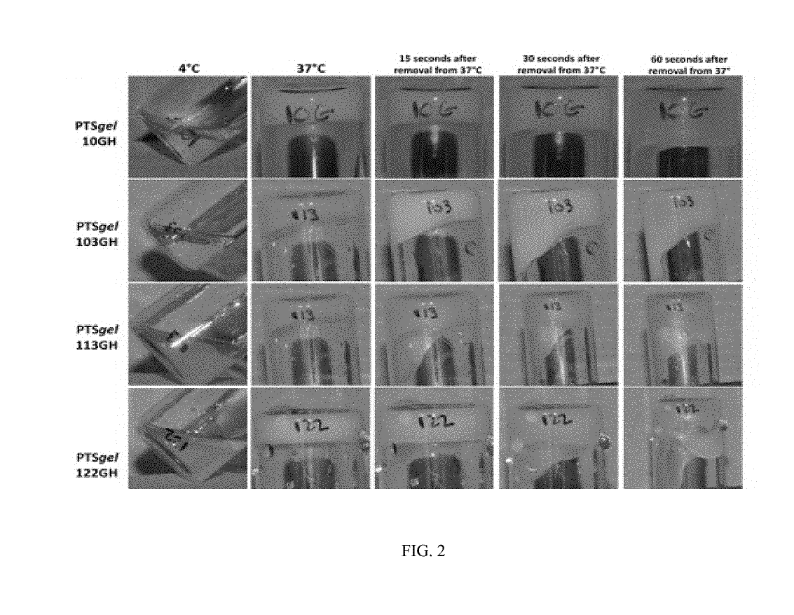

The results of the sol-gel transition analysis are shown in FIG. 2. All polymers were free flowing liquids at 4° C. and at room temperature. The PTSgel 10GH and 103GH were clear liquids whereas, the PTSgel 113GH and 122GH were slightly turbid liquid at room temperature. Immediately after removal of the vials from the 37° C. water bath, the PTSgels were a solid, slightly opaque white gel. On inversion of gel tubes at room temperature, the PTSgels remained a solid gel for ˜25 seconds. Eventually, all gels slowly transitioned back to liquid form at room temperature. All four polymers could be injected through a 31-gauge needle and form a solid gel in PBS at 37° C. Referring to FIG. 2, the sol-gel transition of 10GH, 103GH, 113GH, and 122GH polymers at 4° C. and at room temperature where the PTSgels are in liquid form and transition into solid gels at 37° C. The gels slowly transition back to liquid form at room temperature.

### Example 6—In Vitro Dissolution and Disintegration of PTSgel

In this example the disintegration of PTSgel compositions were analyzed in vitro by two methods: 1. Gravimetric measurement of the residual gelling polymer, and 2. GPC analysis of the residual gelling polymer and supernatant.

First, three concentrations (12.5, 18.75, and 25%) of PTSgel 10GH were evaluated for disintegration/degradation in vitro. Each individual PTSgel (500 uL of 10GH in triplicate) was pipetted into an 8-mL glass vial, weighed, and placed into a 37° C. water bath for gelling for 30 minutes. Four ml of PBS (37° C.) with 0.02% sodium azide as preservative was placed over each PTSgel. The vials were placed into a 37° C. shaker water bath, maintained at 60 rpm. Every 5 days, vials were centrifuged (2000 rpm for 5 minutes at 37° C.), the PBS buffer removed, replaced with fresh PBS, and the vials returned to the 37° C. shaker water bath. Samples of gel were withdrawn after 0, 5, 15, 30, and 45 days and every 15 days thereafter until polymer completely disintegrated. At each time point, vials with residual gel were stored at −80° C. until lyophilized. Vials containing lyophilized gels were weighed and the dry gel weight was determined by subtracting the empty vial weight from the final dry weight.

To measure the gravimetric dry weight loss over time, three concentrations (12.5, 18.75, and 25%) of PTSgel 10GH (500 uL in triplicate) was evaluated for disintegration in phosphate buffered saline (pH 7.4) in vitro at 37° C.

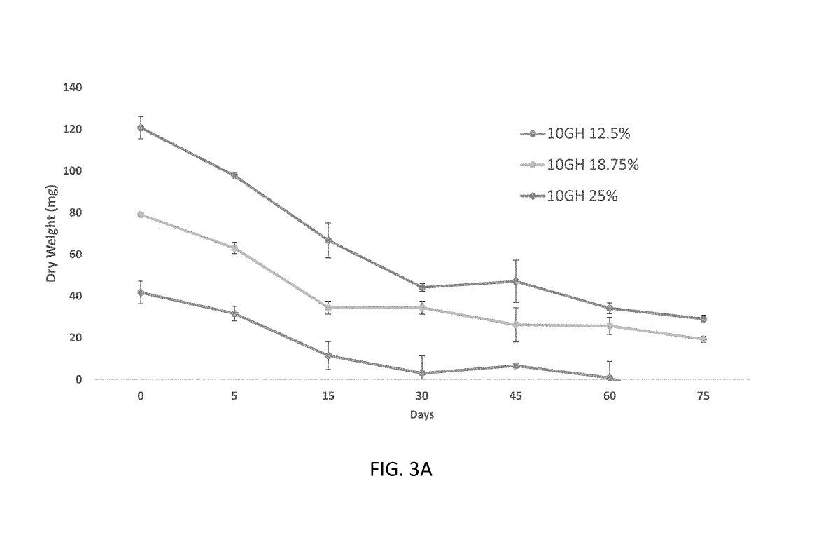

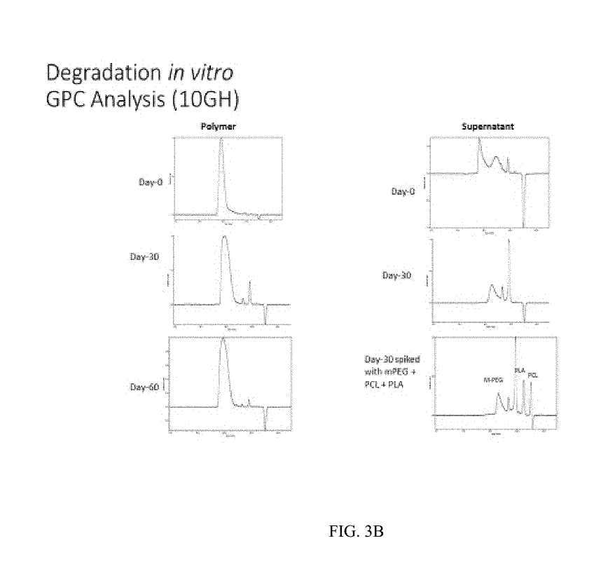

The lowest concentration of 10GH (12.5%) nearly completely disintegrated by 30 days (92.7%) while 18.75 and 25% 10GH PTSgel demonstrated disappearance of gel with 74.2 and 76% dissolved, respectively, by day 75. Polymers are expected to disintegrate faster in vivo compared to in vitro. Lower concentrations of PTSgel were associated with more rapid gel disappearance, with the lowest concentration of 10 GH (12.5%) nearly completely gone by 30 days. The results are shown in FIG. 3A.

Second, the residual of gelling polymer (PTSgel 10GH) and the supernatant collected during the gravimetric studies were analyzed using GPC. The gels were lyophilized and 10-50 mL of the buffer samples were dried using a Genevac EZ-2 evaporator overnight. The lyophilized and dried samples were dissolved in 1 mL of tetrahydrofuran and further dried using 0.3-0.5 g of sodium sulfate. Samples were then vortexed and filtered using 0.2 μm PTFE ISO disc filters. Selected samples were analyzed by GPC as described in Example 3.

Residual polymer and supernatant collected at various time points were analyzed by GPC. It is visually clear that the polymer peaks eluted as a symmetrical peak, unchanged from Day-0 to day-60. The supernatant shows a small amount of dissolved polymer on Day-0, however, there is no polymer present on Day-30 and the disintegrated fragments are much smaller than the polymer but larger than the monomers used for the synthesis. The individual peaks have not been further characterized yet. GPC analysis of residual polymer (10GH, 25%) and supernatant collected for in vitro disintegration was conducted in PBS buffer, pH 7.4 at 37° C. The results are shown in FIG. 3B.

### Example 7—In Vitro Sustained Release Studies from Pentablock Polymer for Hydrophilic and Hydrophobic Molecules

In this example, the sustained release of IgG from PTSgel compositions was analyzed in vitro. In vitro drug release experiments were conducted by adding 500 uL of 22.5% aqueous gelling polymeric solution containing 10 mg of IgG, a large hydrophilic molecule (Human IgG, Lee Biosolutions, Maryland Heights, Mo.) into an 8-mL silanized glass vial (ThermoScientific, Waltham, Mass.) in triplicate. Vials were incubated in a 37° C. water bath for ˜5 minutes until polymer gelled. Release buffer, 4 mL of phosphate buffer saline (PBS, pH 7.4) with 0.02% (wt/vol) sodium azide, was gently layered over the solidified gel in each vial. Vials were sealed with parafilm and maintained at 60 rpm in a 37° C. water bath to replicate physiologic conditions. PBS buffer solution was removed from each vial on days 1, 2, 3, 4, 5, 7, 10, and 14, then weekly thereafter until no more IgG was released. Following collection, 2 mL of fresh 0.02% sodium azide and PBS release buffer (maintained at 37° C.) was layered back into the test vial which was returned to the shaker bath until the next sampling period. The concentration of released IgG samples was evaluated by comparing the released samples to a standard IgG calibration curve. Calibrators and release samples (200 uL) were pipetted into a 96 well, UV-free microplate (Greiner Bio-One, Monroe, N.C.) in triplicate and the absorbance measured at 280 nm (Synergy 2 Microplate Reader, Biotek, Winooski, Vt.). All experiments were set up in triplicate and absorbance of a blank control (0% IgG in 22.5% PTSgel) was subtracted from the released samples for estimation of protein concentration. Similar experiments were set up using three different concentrations of 10GH PTSgel polymer to evaluate further modulation of the release profiles.

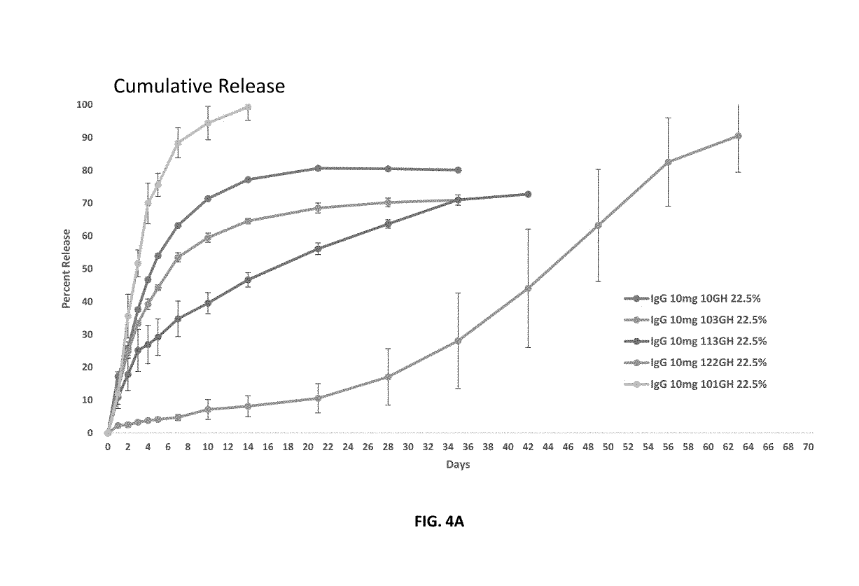

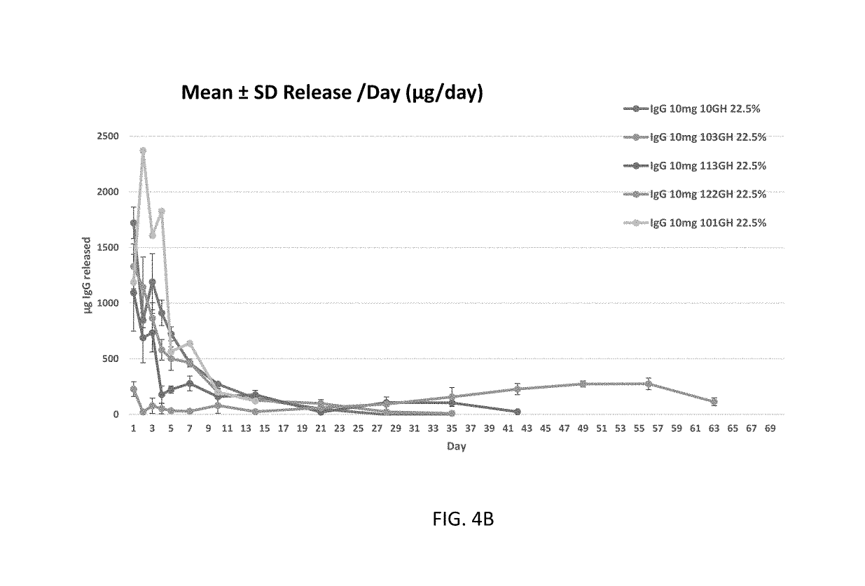

The solid gel PTSgel was maintained at 37° C. and half of PBS buffer was removed and replaced on days 1, 2, 3, 4, 5, 7, 10, 14, and then weekly thereafter until no more release was observed. Extensive modulation of in vitro release was achieved by using five different PTSgels; 101GH, 10GH, 103GH, 113GH and 122GH, with increasing hydrophobicity of the polymers in the order respectively. There was a low initial burst of drug release at 20 mg/mL IgG (2.3-17.2% release on day 1) with most polymers and almost negligible with the 122GH, the most hydrophobic polymer tested (2.3%) which is the most hydrophobic of all polymers. Initial release is followed by a controlled, release over extended period of several days to weeks as shown in FIG. 4A. Using 22.5% of each PTSgel, the quickest release rate was observed with the most hydrophilic polymer in the group (101GH) with ˜99% of the IgG load released in 14 days and the remaining released at an intermediate rate. The PTSgel 10GH and 103GH, had a slower release with 80% and 69% of the IgG released in 21 days respectively. Referring to FIG. 4A, both the PTSgel 113GH and 122GH had a much slower rate of release with 73% and only 44% by day-42 respectively. From 122GH (the most hydrophobic polymer), released 91% of IgG by day-63 whereas 99% of the release was achieved in ˜14 days from the most hydrophilic polymer (101GH). A significant decrease in initial burst release related to increase in hydrophobicity demonstrates that the capacity to hold drugs increases with increase in hydrophobicity of these PTSgels. Further, referring to FIG. 4B, the modulation of drug release at different rates can be achieved for long periods by changing the hydrophobicity of the polymers. The more hydrophobic the polymer, the drug was released at a slower rate and thus lasted for much longer duration. Overall, modulation of in vitro release was observed by differing rates of IgG release from the 5 tested PTSgels. There was a controlled, steady release rate over extended period of days to several weeks. Polymer 122GH demonstrated minimal initial burst of drug release.

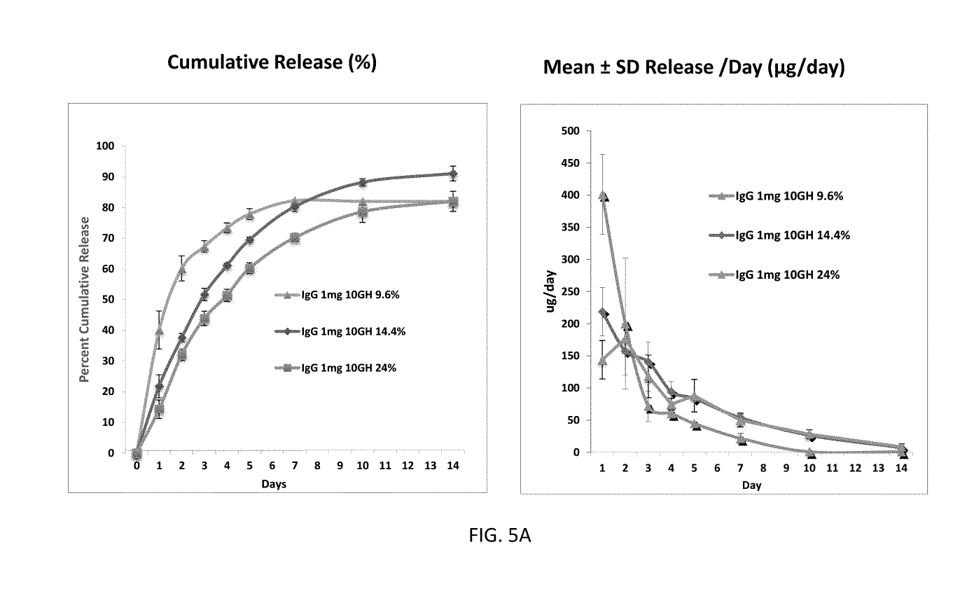

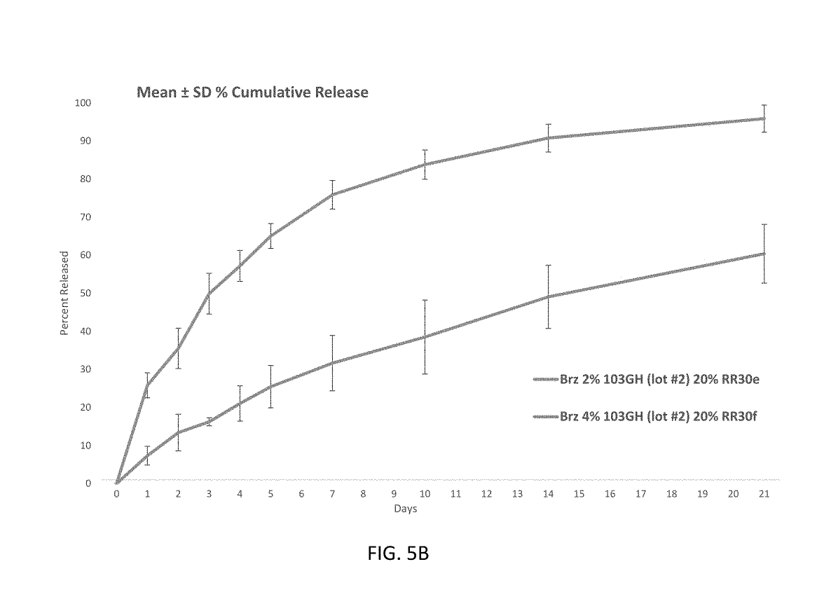

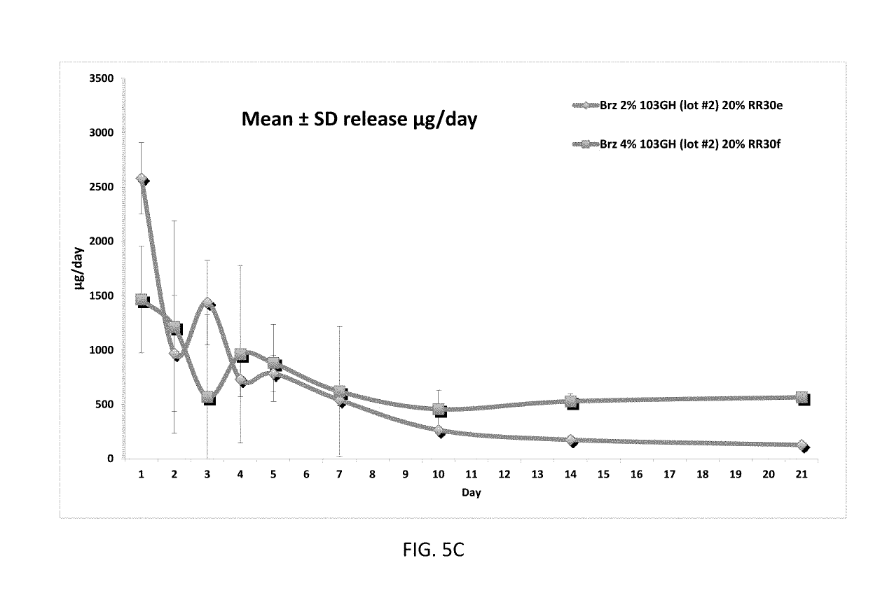

Referring to FIG. 5A, the initial burst is also reduced considerably when lower drug concentration (1 mg/mL) IgG concentrations were loaded into the 10GH PTSgel. Again, demonstrating that the polymer's capacity to hold the drug dictates initial burst release. The initial burst is only seen when drug loading is higher than the loading capacity of the individual polymer. In addition, in vitro release of IgG was also demonstrated to be modulated by varying the concentration of PTSgel polymer. Using 11 mg/mL IgG in 9.6% 10GH PTSgel, resulted in a faster release of IgG, accompanied by a higher (40%) initial release on day 1 and 80% cumulative release by day 7, compared to a gradual and more sustained release achieved with 14.4 and 24% of 10GH PTSgel and less initial burst, again suggesting that the drug release is a function of polymer capacity which increases with higher polymer concentration. The results are shown in FIG. 5 A.

FIGS. 5B and 5C show the release profile for brinzolamide, a hydrophobic small molecule (2% and 4%) from PTS 103GH. It also illustrates that drug release modulation can also be achieved by changing drug concentration.

### Example 8—Characterization of IgG Structural Integrity Released from PTSgel

In this example, the structural integrity of IgG released from PTSgel compositions were analyzed using two methods: 1. Sodium Dodecyl Sulfate Polyacrylamide Gel Electrophoresis (SDS-PAGE) analysis under reducing and non-reducing conditions, and 2. size exclusion High Performance Liquid Chromatography (HPLC) analysis using a Shodex column.

First, collected buffer samples from the in vitro release assays were evaluated for IgG integrity by SDS-PAGE analysis. The IgG samples included were standards and the samples released in PBS buffer (pH 7.4, 37° C.) after incorporation into a selected PTSgel. Samples were evaluated within 7 days of collection and stored at 4° C. until analysis. IgG standards, diluted in PBS, or sample eluates were combined with 4× Laemmli dye, with (reducing) or without (non-reducing) β-ME, to achieve a 1× dye concentration. Reduced samples were heated to 95° C. for 10 minutes, cooled, and loaded on 4-12% Bis-Tris NuPAGE gels (Life Technologies, Carlsbad, Calif.). Pre-stained markers (cat. no. LC5925, Life Technologies) and non-reduced samples were loaded without heating. Gels were run in MOPS buffer (Life Technologies), with antioxidants for reduced samples only, at 180V for 60 minutes. Electrophoresed gels were fixed in 50% methanol, 10% acetic acid for 30 minutes, stained in Coomassie Brilliant Blue R250 Staining Solution (Bio-Rad Laboratories, Inc., Hercules, Calif.) for 30 minutes, and destained in 5% methanol, 7.5% acetic acid until clear. Gels were scanned on a Canon CanoScan 9000F at 600 dpi and images saved as TIFFs in Adobe Photoshop.

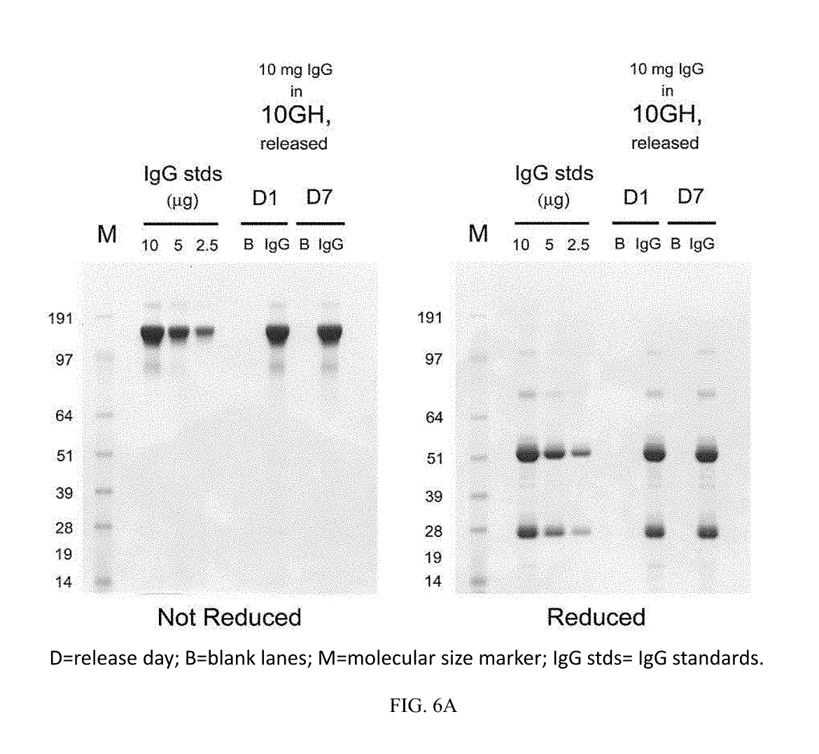

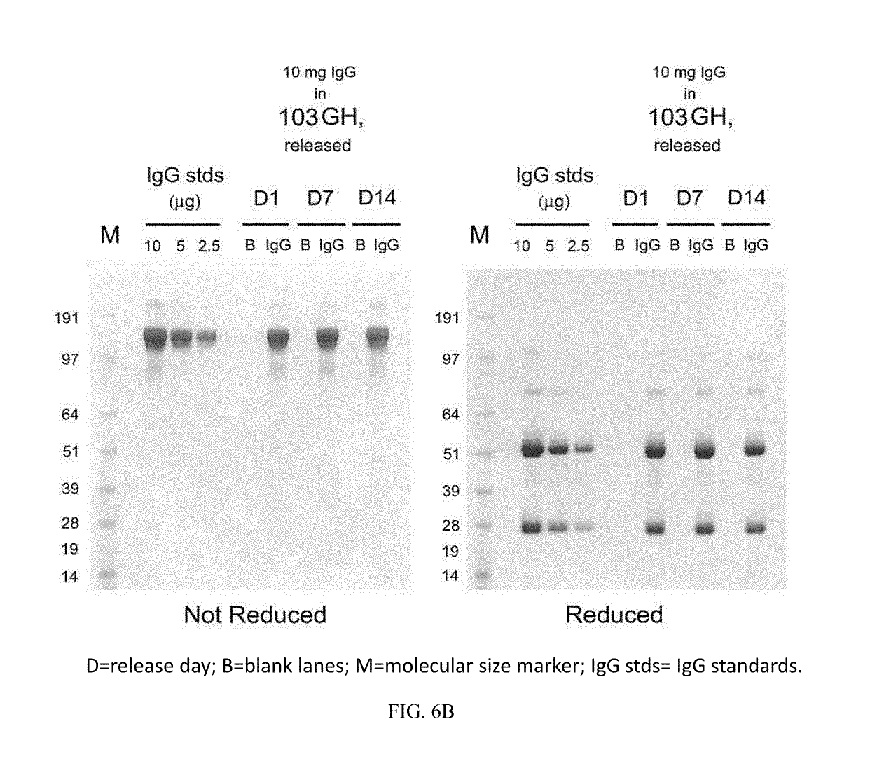

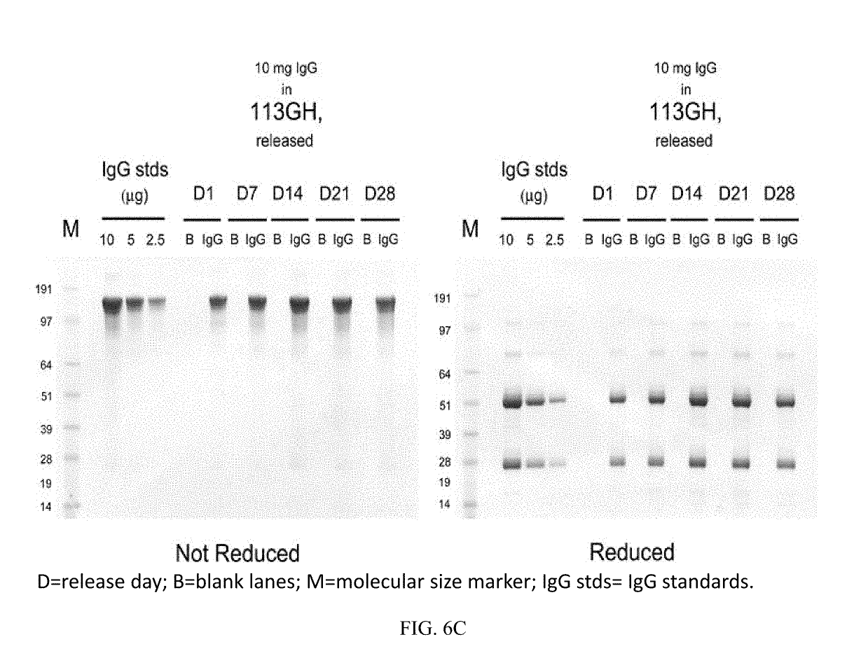

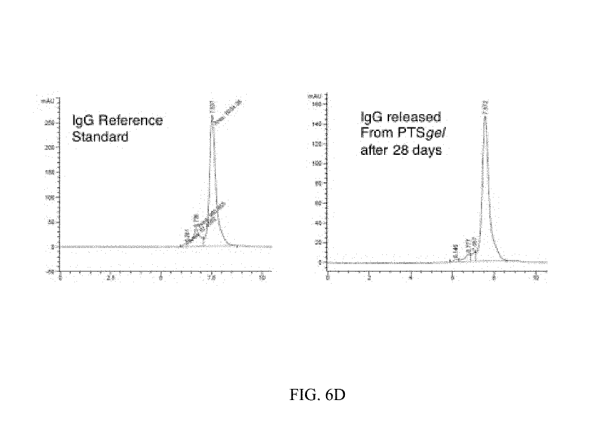

Reduced (using beta-mercaptoethanol) and non-reduced sodium dodecyl sulfate-polyacrylamide gel electrophoresis (SDS-PAGE) was used for size-based separations of the IgG for determining IgG integrity in the in vitro samples released from PTSgels over a period of 7 to 28 days. Reducing conditions disrupt disulphide bonds separating IgG light and heavy chains. The IgG released from 10GH had the same bands of approximately 150 kDa for the non-reduced and 28 and 51 kDa for the reduced SDS-PAGE at days 1 and 7, which appeared identical to the IgG standards (FIG. 6A). These results suggest that the IgG molecules were intact and no degradation of IgG had occurred during incubation with PTSgel and after release into the buffer. Similarly, there appeared to be excellent integrity for the IgG released from the 103GH polymer through 14 days (FIG. 6B) and the 113GH polymer through 28 days (FIG. 6C), suggesting that the PTSgel polymer did not affect the integrity of the IgG protein.

Second, collected buffer samples from the in vitro release assays were evaluated for IgG integrity by SE-HPLC analysis. The IgG samples included were standards and the samples released in PBS buffer (pH 7.4, 37° C.) after incorporation into a selected PTSgel for up to 28 days (FIG. 6D). Samples were stored at −20° C. until analysis. Twenty microliters of IgG standards, diluted in PBS, or sample eluates were analyzed on a Shodex column (KW403-4F, 4.6 mm ID×300 mm L) using a UV absorption detector with the wavelength selected at 214 nm. The mobile phase was 100 mM sodium phosphate, 250 mM NaCl, pH 7.0 used under isocratic conditions at 0.35 mL/min.

Referring to FIG. 6D, compare chromatograms of SE-HPLC analysis conducted on a reference standard IgG and on an in vitro sample released after incubation with 10GH for 28 days at 37° C. IgG maintained its structural integrity as is clearly demonstrated in FIG. 6D.

### Example 9—In Vivo Sustained Release Studies from Pentablock Polymer

In this example, the sustained release of IgG from pentablock polymer compositions was studied in vivo. IgG was labeled with a near-infrared (NIR) dye (IRDye800CW) by LICOR Biosciences, Lincoln, Nebr. NIR-labeled IgG in PTSgel gelling solution or in PBS was made by adding 1 ml of cold PTSgel (25% polymer solution in PBS, pH 7.4) or PBS to 1 mg of lyophilized NIR-labeled IgG. After gentle vortexing, the solutions were stored at 4° C. until used within 24 hours. Insulin syringes with 31G needles were used to inject 200 uL of solution subcutaneously over the dorsum of a CD-1 mouse (Charles River, Morrisville, N.C.) that was maintained on an alfalfa-free diet. Mice (n=3) were injected with PBS, PTSgel, or a combination of NIR-IgG in PBS or NIR-IgG in PTSgel. Mice were anesthetized with 2.5% isoflurane in oxygen and imaged using an in vivo imager (IVIS, Xenogen, Alameda, Calif.) using Indocyanine Green (ICG) settings. Quantification of fluorescence was measured using the imaging software automated region of interest (ROI) setting to calculate the radiant efficiency of the injection site. Mice were imaged prior to injection, immediately after injection, then post-injection on days 1-5, 7, 10 and 14, and then weekly using the same imager settings and protocol as used for Day-0 imaging.

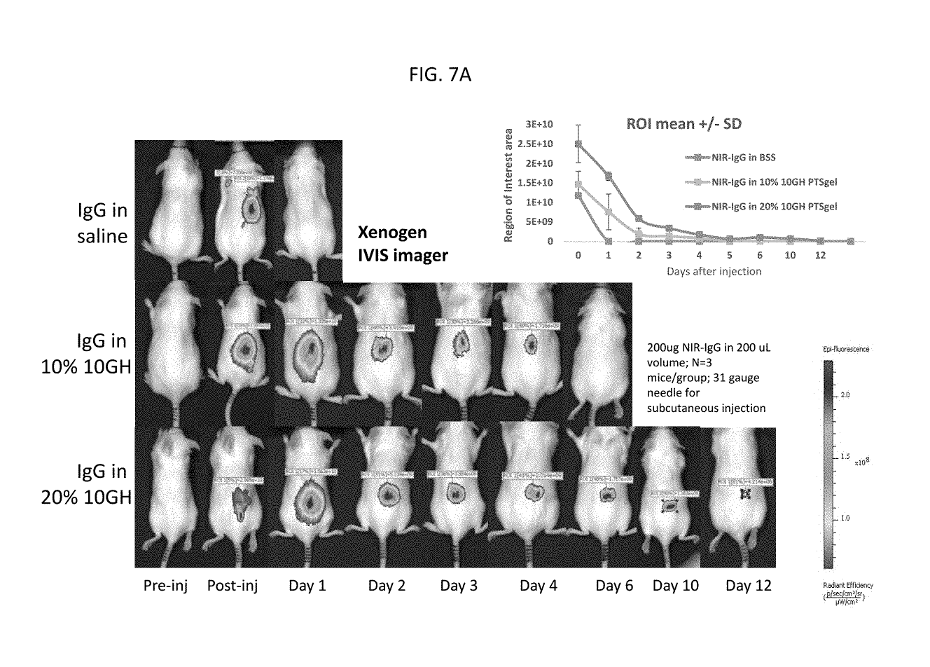

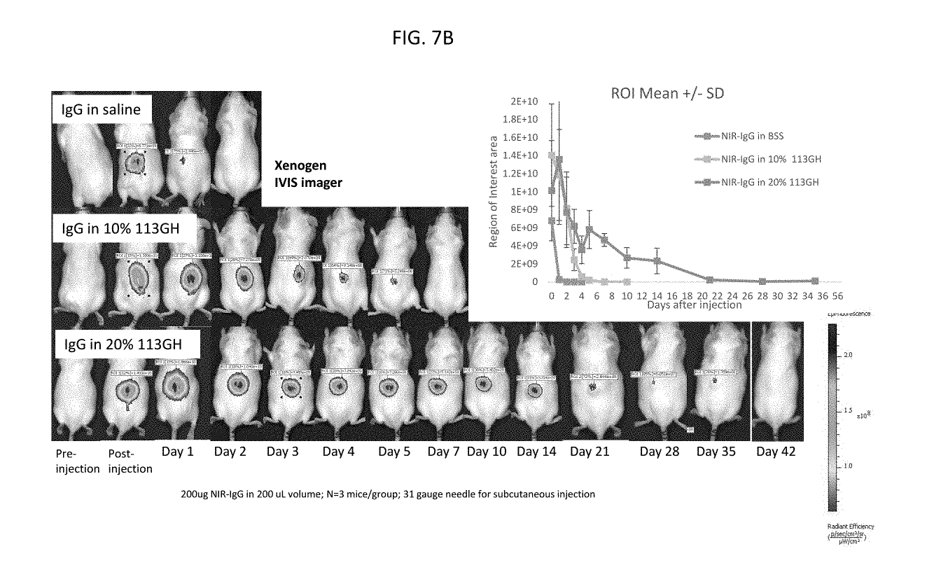

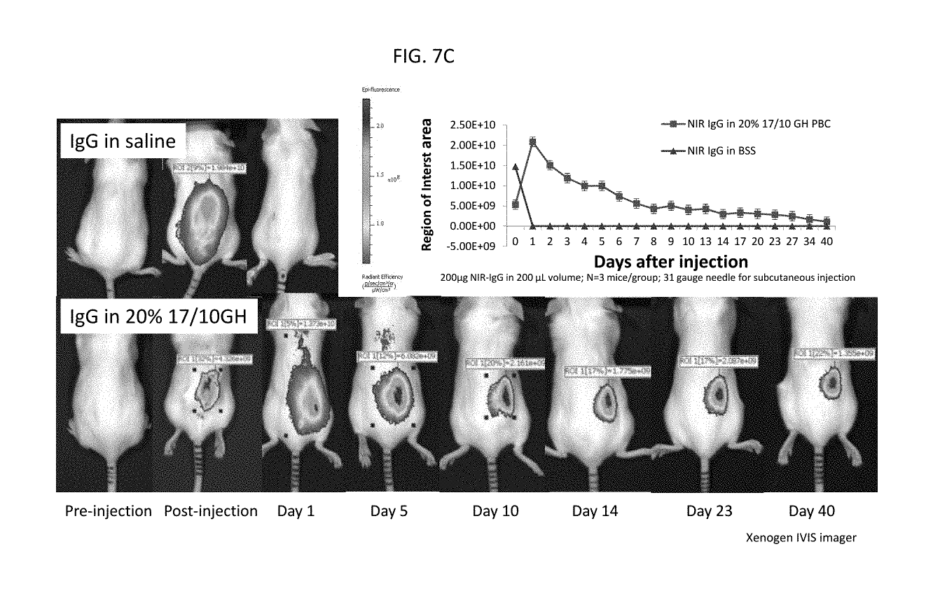

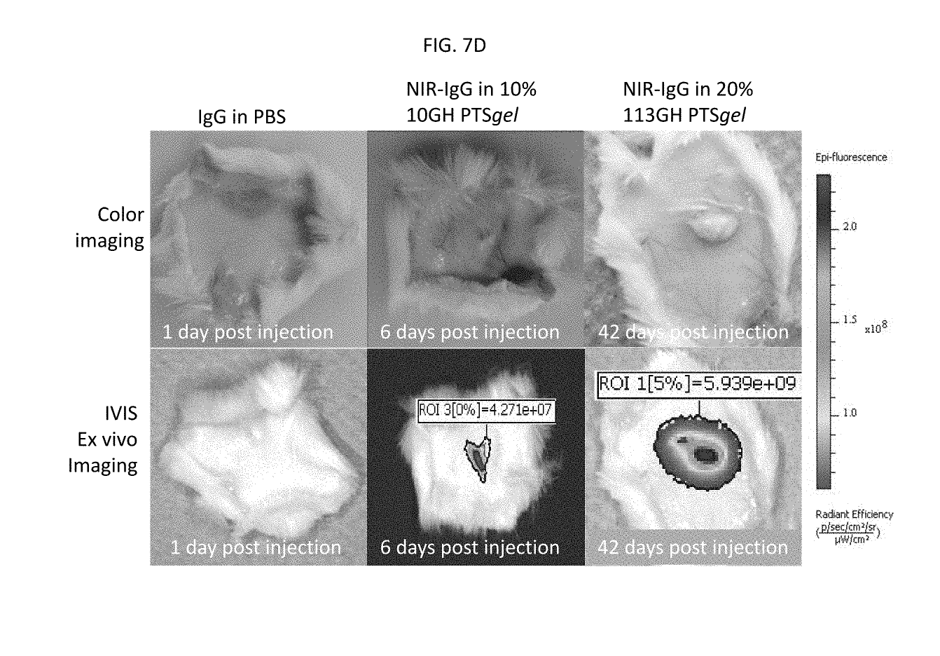

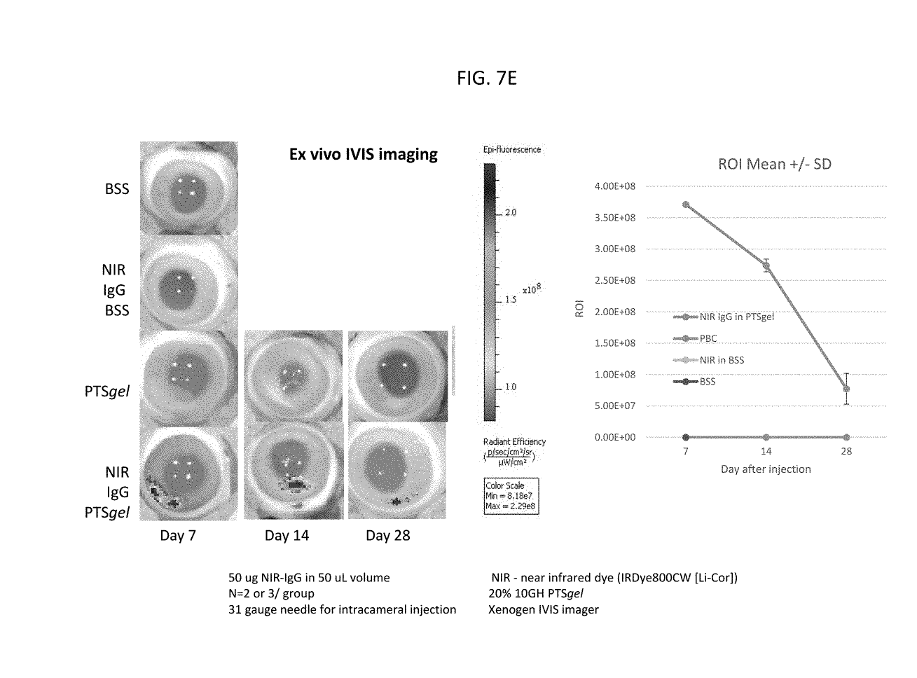

Following subcutaneous injection of 200 uL of PTSgel (either 10GH or 113GH) containing 200 ug of near-infrared dye labeled IgG (NIR-IgG) or NIR-IgG in PBS in mice, no adverse reaction, swelling, or redness was observed at the injection site for the duration of the study. Referring to FIGS. 7A-7B, NIR-IgG in PBS was visible on IVIS imaging immediately after injection, but by 24 hours after injection, no NIR fluorescence was visible. Fluorescence of NIR-IgG in the PTSgel in vivo paralleled that observed in the in vitro release rates for 10GH and 113GH presented earlier in Example 7. Referring to FIG. 7A, mice injected with 10% 10GH had fluorescence through approximately day 4, while those injected with 20% 10GH fluoresced through approximately day 14. Referring to FIG. 7B, mice injected with 10% 113GH fluoresced through approximately 7 days, and those with 20% 113GH fluoresced through approximately 35 days.

A similar sustained release experiment was performed by making a subcutaneous injection of labeled IgG (NIR-IgG) in a mixture of two polymers (PTS10 GH and PTS 17GH, mixed in 1:1 ratio and at 20% final polymer concentration) and was compared against NIR-IgG in PBS in mice. FIG. 7C illustrates in vivo IVIS imaging and quantitative profiles in mice.

Referring to FIG. 7D, once the mice were negative for fluorescence on IVIS imaging, they were euthanized, the skin at the site of the injection excised, and imaged ex vivo. In all animals, there was a very small deposit of gel visible in the subcutaneous tissue suggesting nearly complete dissolution/disintegration of the PTSgel. On imaging, there was a small signal of fluorescence that corresponded to the size of the gel deposit, suggesting that IgG was tightly held by the PTSgel and the rate of IgG release and gel dissolution/disintegration were parallel and that an “empty shell” of undissolved PTSgel did not remain

Further, following ocular anterior chamber injection of 50 uL of 20% 10GH PTSgel containing 50 ug of near-infrared dye labeled IgG (NIR-IgG) or NIR-IgG in PBS in rabbits eyes were imaged ex vivo using IVIS imaging. Referring to FIG. 7E, rabbits injected with NIR-IgG in 20% 10GH fluoresced through approximately day 28, while NIR-IgG in PBS was not visible by 24 hours after injection. A small deposit of gel was visible in the ocular tissue at day 28, suggesting a nearly complete dissolution/disintegration of the PTSgel.

### Example 10—In Vivo Safety Assessment of PTSgel Compositions

In this example, the in vivo safety of PTSgel compositions was assessed using subcutaneous injections. Assessment of the injection site was done at each imaging time to evaluate for signs of inflammation or swelling. Once the injection site was negative for dye detection on IVIS imaging, the mice were euthanized and the skin at the injection site collected, the inverted skin exposing the injection site/PTSgel depot was imaged ex vivo using IVIS imaging to detect residual IgG, and the skin section was fixed in 10% formalin. The formalin-fixed skin was then processed for histopathology, stained with hematoxylin and eosin, and examined using light microscopy.

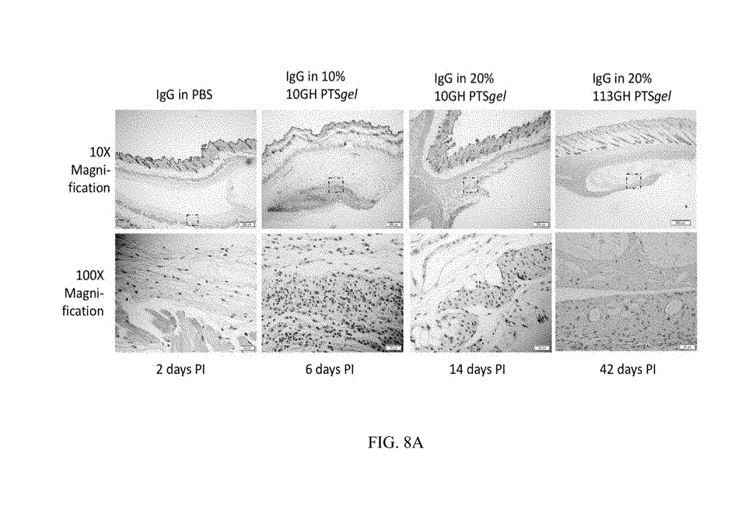

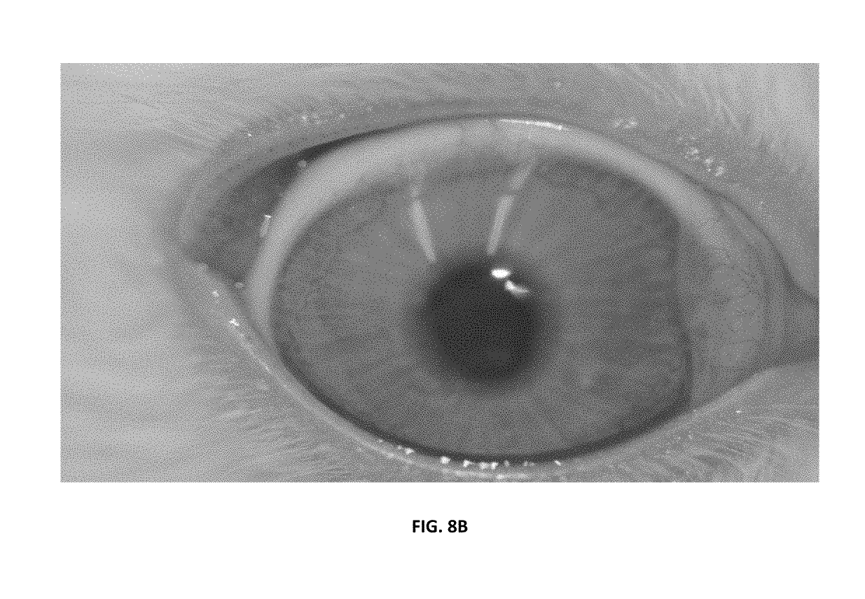

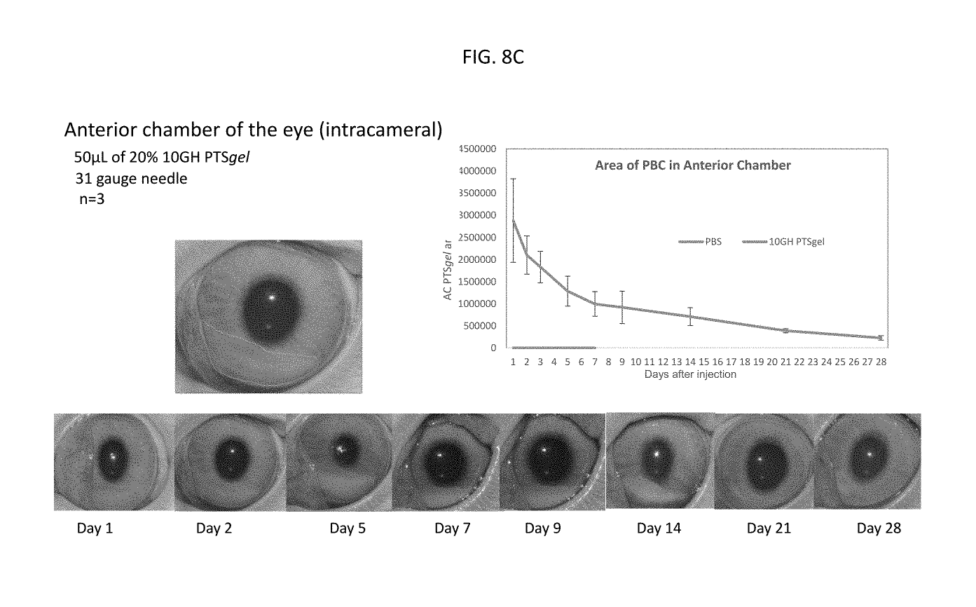

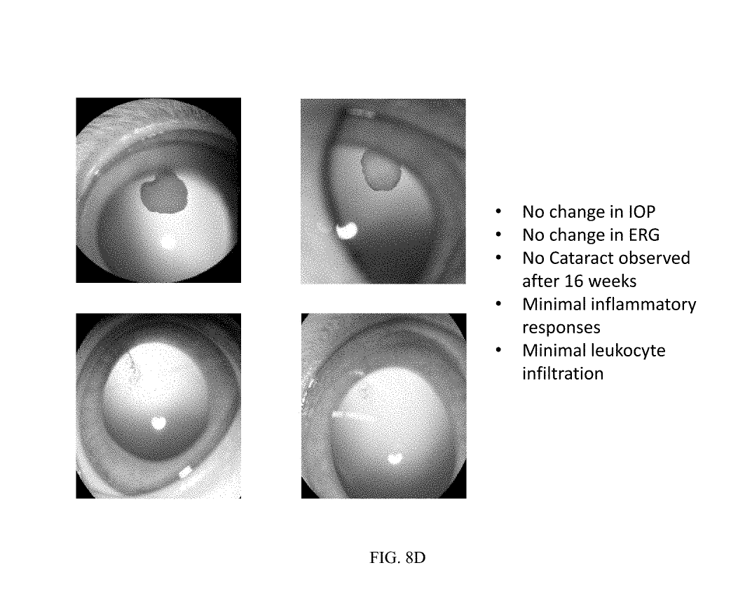

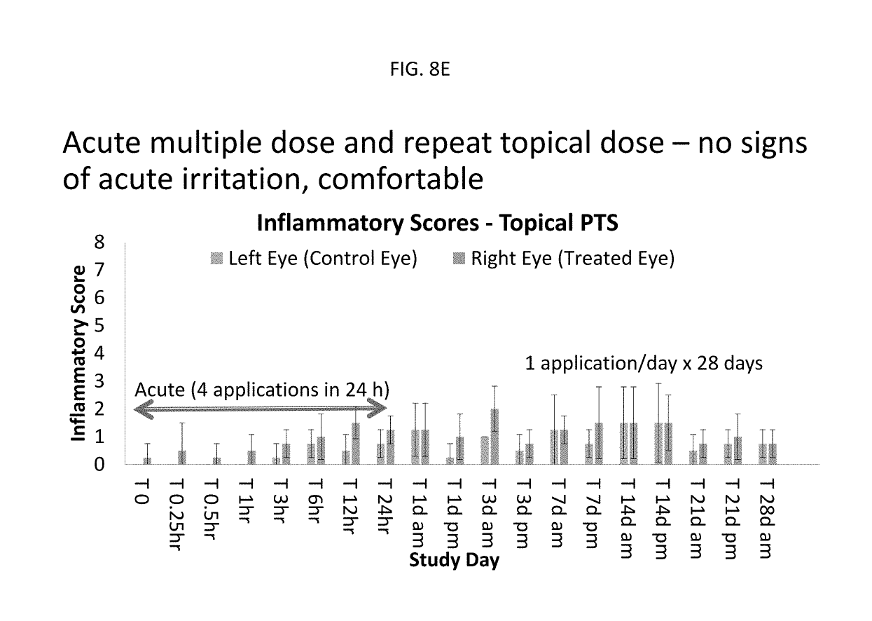

Following imaging of the skin samples, they were fixed and processed for histopathology. Referring to FIG. 8A, using low magnification light microscopy (upper row of images), the subcutaneous depot of PTSgel was visualized in the 10 and 20% 10GH and 113GH-injected tissue. No inflammation or subcutaneous depot was visualized in animals injected with NIR-IgG in PBS. On higher magnification (lower row of images), the 10GH PTSgel at 6 days had a mild infiltrate of and mononuclear cells (e.g., scattered neutrophils and macrophages) surrounding and infiltrating the site of the injection but in the 14 (20% 10GH) and 42 day (20% 113GH) there were macrophages surrounding the depot but no epidermal or dermal inflammation or swelling observed. The dotted box in the upper row of images was the site of higher magnification represented in the lower row of images.

Further, following topical administration, ocular anterior chamber, or intravitreal injection in rabbits, no adverse reaction, swelling, or redness was observed at the injection site for the duration of the study. Referring to FIG. 8B, the ocular intravitreal injection of 50 μl PTSgel was monitored for 1, 21, 42, and 49 days. The injection was well tolerated with no signs of changes in intraocular pressure or electroretinography, no cataract formation at 16 weeks, with minimal inflammatory response and minimal leukocyte infiltration observed. Referring to FIG. 8B, the degradation of a 20% 10GH PTSgel composition was monitored after injecting 50 μl of the composition into the anterior chamber of the eye for 21 days. The level of degradation was determined by the area of the composition in the anterior gel measured at days 1, 2, 5, 7, 9, 14, and 21. The level of degradation of the composition was much faster in vivo than in vitro. Referring to FIG. 8C, the topical administration of PTSgel was well tolerated 30 minutes after application. Referring to FIG. 8D, chronic topical application was studied by administering 35 μL of PTSgel was administered topically 4 doses, 15 minutes apart for the first day (Acute), then twice a day (6 hours apart) for 28 days (repeat dose). Inflammation scores remained low and no signs of irritation or discomfort were reported throughout the duration of the topical administration study.

### Example 11—Therapeutic PTSgel Compositions

In this example, drugs which are commercially only available as emulsions or suspensions have been successfully tested for sustained released using PTSgels. The drugs are initially dissolved/suspended in the neat PTSgel polymers followed by the addition of PBS buffer, pH 7.4 to make aqueous solution of polymers and drugs dissolved in. Drugs can be added at much higher concentration than what is commercially available only as emulsions or suspensions. Most drugs completely dissolve but some may dissolve only partially. However, the polymer dispersions containing the drugs are liquid at room temperature and gel at body temperature and demonstrate sustained release of these hydrophobic drugs in vitro. Exemplary drugs such as, brinzolamide, difluprednate, colecoxib, pazopanib and cyclosporine, can be incorporated into PTSgel compositions.

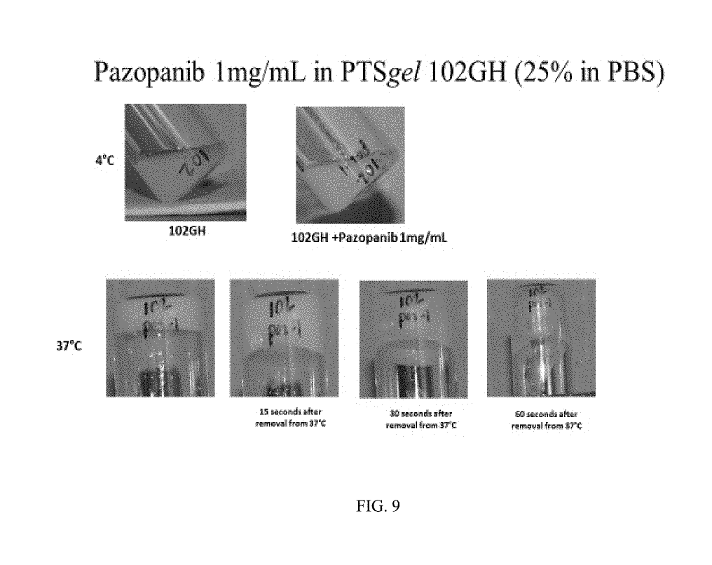

Referring to FIG. 9, a solution comprising 102GH PTSgel (25% polymer in PBS) with pazopanib is shown at 4° C. and 37° C. temperatures. The 102GH PTSgel (25%) with 1 mg/mL pazopanib is demonstrated to gel at 37° C. and a solution at 4° C.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for the use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items. “Consisting essentially of” means inclusion of the items listed thereafter and which is open to unlisted items that do not materially affect the basic and novel properties of the disclosure.

### INCORPORATION BY REFERENCE

All publications and patents mentioned herein are hereby incorporated by reference in their entireties as if each individual publication or patent was specifically and individually indicated to be incorporated by reference.

1. A composition for sustained release of an active ingredient, comprising a co-block polymer having the formula of PEG-PCL-PLA-PCL-PEG or PGA-PCL-PEG-PCL-PGA in the form of a gel formulation,
wherein PEG is polyethylene glycol and has an average molecular weight of about 100 to about 1000 Da, preferably about 350 to about 750 Da, more preferably about 400 to about 550 Da;
wherein PCL is poly(ε-caprolactone) and has an average molecular weight of about 100 to about 3000 Da, preferably about 200 to about 2000 Da, more preferably about 400 to about 1500 Da;
wherein PLA is polylactic acid and has average molecular weight of about 100 to about 5,000 Da, preferably about 150 to about 1,500 Da, more preferably about 250 to about 1,100 Da; and
wherein PGA is polyglycolic acid and has average molecular weight of about 100 to about 5,000 Da, preferably about 500 to about 1,500 Da, more preferably about 250 to about 1,100 Da.

2. The composition of claim 2, wherein the PEG, PCL, PLA and/or PGA are present in an amount to increase hydrophobicity of the block polymer, thereby achieving tunable sustained release of the active ingredient, irrespective of the hydrophilicity or hydrophobicity of the active ingredient.

3. The composition according to claim 1, wherein the co-block polymer has a total molecular weight of about 1500-10,000 Da, preferably about 2000-7000 Da, and more preferably about 2500-5000 Da.

4. The composition according to claim 1, wherein the composition bio-degrades in vivo in substantially similar time required for the release of the active ingredient, allowing for repeat injections.

5. The composition according to claim 1, further comprising an aqueous medium and an active ingredient admixed therein, wherein the polymer is present at between about 1 wt % and about 50 wt %, said composition showing sustained release of the active ingredient in vitro and in vivo.

6. The composition according to claim 5, wherein the polymer is present at between about 5 wt % and about 40 wt %.

7. The composition according to claim 5, wherein the polymer is present at between about 10 wt % and about 30 wt %.

8. The composition according to claim 5, wherein the active ingredient is present at between about 0.01 wt % and about 50 wt %.

9. The composition according to claim 5, wherein the active ingredient is a biologic or chemical agent.

10. The composition according to claim 9, wherein the active ingredient is hydrophobic or hydrophilic, or a mixture of hydrophobic and hydrophilic ingredients.

11. The composition according to claim 1, comprising two or more co-block polymers.

12. A method of delivering an active ingredient to a mammal in need thereof, comprising:
providing the composition of claim 1 admixed with an active ingredient, wherein the polymer is present at between about 1 wt % and about 50 wt %,
administering the composition to a mammal by a parenteral route or topical application.

13. The method of claim 12, further comprising extending release time of biologically active levels of the active ingredient longer than a standard vehicle.

14. The method of claim 12, wherein the polymer bio-degrades at a release rate substantially similar to an active ingredient, allowing for repeat applications without interfering biologically or physically with a prior application.

15. The method of claim 12, wherein the polymer biodegrades successively into substituent blocks, which are not substantially physiologically harmful, and wherein the polymer and the substituent blocks from biodegradation are tolerated in vivo such that long-term or repeat applications are feasible.

16. The method of claim 12, wherein the active ingredient retains its biologic activity over the entire course of release of up to 6 months or longer.
